SEQUENCE LISTING (8 sequences)

SEQ ID NO: 1 - DNA, 1491 bp, synthetic construct
  source 1..1491: mol_type = other DNA
gatgtcgtgatgactcagagccccgattccctggccgtgtccctgggcgagagagccacaatcaactgcaagagctcccagagcctgctggactctgatggcaagacctacctgaattggctgcagcagaagccaggccagcctccaaagcggctgatcagcctggtgtccaagctggatagcggcgtgcctgacagattcagcggctccggcagcggcaccgactttaccctgacaatcagctctctgcaggccgaggatgtggccgtgtattactgctggcagggcacccactttcctggcacctttggcggcggcaccaaggtggagatcaagggcggcggcggctctggcggcggcggctctggcggcggcggcagcggcggcggcggcagcgagatccagctggtgcagtccggcgccgaggtgaagaagcccggcgagtccctgagaatctcctgcaagggctccggctttaatatcgaggactactacatccactgggtgcggcagatgccaggcaagggcctggagtggatgggcagaatcgatccagagaacgatgagaccaagtacggcccaatcttccagggccacgtgacaatcagcgccgatacaagcatcaataccgtgtatctgcagtggtcttccctgaaggcctccgataccgccatgtattactgcgccttcagaggcggcgtgtattggggccagggcaccacagtgacagtgtccagcggcggcggcggctctgaggtgcagctggtggagtctggcggcggcctggtgcagcccggcggcagcctgagactgtcttgcgccgccagcggctattccttcacaggctataccatgaactgggtgcggcaggcccctggcaagggcctggagtgggtggccctgatcaacccatataagggcgtgtccacctacaaccagaagttcaaggaccgctttaccatctctgtggacaagagcaagaatacagcctacctgcagatgaacagcctgagagccgaggatacagccgtgtactattgcgccagatccggctattacggcgactccgactggtacttcgacgtgtggggccagggcacactggtgaccgtgtcttccggcggcggcggcagcggcggcggcggctccggcggcggcggctccgacatccagatgacccagtccccatcttccctgtccgcctctgtgggcgatagagtgacaatcacctgccgggcctctcaggacatccggaattatctgaactggtaccagcagaagcccggcaaggcccctaagctgctgatctactataccagcaggctggagagcggcgtgccatccagattctccggcagcggctctggcacagactataccctgaccatctccagcctgcagccagaggattttgccacctattactgtcagcaggggaacactctgccttggacattcggccagggaacaaaagtggaaatcaaaagtagc

SEQ ID NO: 2 - protein, 497 aa, synthetic construct
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 3 - DNA, 1545 bp, synthetic construct
  source 1..1545: mol_type = other DNA
atggactggacctggattctgttcctggtggcagccgctacaagagtgcattccgatgtcgtgatgactcagagccccgattccctggccgtgtccctgggcgagagagccacaatcaactgcaagagctcccagagcctgctggactctgatggcaagacctacctgaattggctgcagcagaagccaggccagcctccaaagcggctgatcagcctggtgtccaagctggatagcggcgtgcctgacagattcagcggctccggcagcggcaccgactttaccctgacaatcagctctctgcaggccgaggatgtggccgtgtattactgctggcagggcacccactttcctggcacctttggcggcggcaccaaggtggagatcaagggcggcggcggctctggcggcggcggctctggcggcggcggcagcggcggcggcggcagcgagatccagctggtgcagtccggcgccgaggtgaagaagcccggcgagtccctgagaatctcctgcaagggctccggctttaatatcgaggactactacatccactgggtgcggcagatgccaggcaagggcctggagtggatgggcagaatcgatccagagaacgatgagaccaagtacggcccaatcttccagggccacgtgacaatcagcgccgatacaagcatcaataccgtgtatctgcagtggtcttccctgaaggcctccgataccgccatgtattactgcgccttcagaggcggcgtgtattggggccagggcaccacagtgacagtgtccagcggcggcggcggctctgaggtgcagctggtggagtctggcggcggcctggtgcagcccggcggcagcctgagactgtcttgcgccgccagcggctattccttcacaggctataccatgaactgggtgcggcaggcccctggcaagggcctggagtgggtggccctgatcaacccatataagggcgtgtccacctacaaccagaagttcaaggaccgctttaccatctctgtggacaagagcaagaatacagcctacctgcagatgaacagcctgagagccgaggatacagccgtgtactattgcgccagatccggctattacggcgactccgactggtacttcgacgtgtggggccagggcacactggtgaccgtgtcttccggcggcggcggcagcggcggcggcggctccggcggcggcggctccgacatccagatgacccagtccccatcttccctgtccgcctctgtgggcgatagagtgacaatcacctgccgggcctctcaggacatccggaattatctgaactggtaccagcagaagcccggcaaggcccctaagctgctgatctactataccagcaggctggagagcggcgtgccatccagattctccggcagcggctctggcacagactataccctgaccatctccagcctgcagccagaggattttgccacctattactgtcagcaggggaacactctgccttggacattcggccagggaacaaaagtggaaatcaaaagtagc

SEQ ID NO: 4 - protein, 515 aa, synthetic construct
MDWTWILFLVAAATRVHSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 5 - DNA, 1527 bp, synthetic construct
  source 1..1527: mol_type = other DNA
atggactggacctggatactgttcctggtggccgccgccacacgggtgcactccgacatccagatgacccagtcccctagctccctgagcgcctccgtgggcgatagagtgacaatcacctgtaaggccagccaggacgtgtccatcggcgtggcctggtaccagcagaagccaggcaaggcccccaagctgctgatctacagcgcctcctataggtataccggcgtgccatcccgcttttctggctctggctccggcacagacttcaccctgacaatctccagcctgcagccagaggacttcgccacatactattgccagcagtattacatctacccctacaccttcggccagggcaccaaggtggagatcaagagaggcggcggcggctctggcggcggcggcagcggcggcggcggctccgaggtgcagctggtggagtctggcggcggcctggtgcagccaggcggctctctgcggctgagctgtgccgcctccggcttcaccttcacagattacaccatggactgggtgcgccaggcccccggcaagggcctggagtgggtggccgatgtgaatcccaatagcggcggctctatctataaccagcggtttaagggccggtttaccctgtctgtggaccgcagcaagaataccctgtacctgcagatgaacagcctgcgcgccgaggataccgccgtgtactactgcgccagaaacctgggccctagcttctactttgattactggggccagggcaccctggtgacagtgtccagcggcggcggcggcagcgaggtgcagctggtggagtccggcggcggcctggtgcagcctggcggcagcctgagactgtcctgcgccgcctccggctactctttcacaggctataccatgaattgggtgcggcaggccccaggcaagggcctggagtgggtggccctgatcaacccctataagggcgtgtccacatacaaccagaagttcaaggacaggtttaccatctccgtggataagtccaagaataccgcctacctgcagatgaattccctgcgggccgaggatacagccgtgtactattgcgcccgctccggctattacggcgatagcgactggtatttcgacgtgtggggccagggcacactggtgacagtgagctccggcggcggcggctccggcggcggcggctctggcggcggcggctccgatatccagatgacccagtctccctccagcctgtccgcctctgtgggcgaccgggtgaccatcacctgtcgcgccagccaggatatccggaactatctgaactggtatcagcagaagcccggcaaggccccaaagctgctgatctactatacctctagactggagagcggcgtgccttccaggttttccggctctggcagcggcaccgactataccctgaccatctcctctctgcagccagaggatttcgccacatattattgtcagcagggcaataccctgccctggacatttggccagggcacaaaggtggagatcaag

SEQ ID NO: 6 - protein, 491 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 7 - DNA, 1527 bp, synthetic construct; identical to SEQ ID NO: 5
  source 1..1527: mol_type = other DNA

SEQ ID NO: 8 - protein, 509 aa, synthetic construct
MDWTWILFLVAAATRVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

(19) United States
(12) Patent Application Publication
(10) Pub. No.: US 2025/0345354 A1
(43) Pub. Date: Nov. 13, 2025

(54) COMBINATION OF DNA-ENCODED BISPECIFIC T-CELL ENGAGERS TARGETING CANCER ANTIGENS AND METHODS OF USE IN CANCER THERAPEUTICS

(71) Applicant: The Wistar Institute of Anatomy and Biology, Philadelphia, PA (US)

(72) Inventors: David Weiner, Merion, PA (US); Daniel Park, Philadelphia, PA (US)

(21) Appl. No.: 18/869,620

(22) PCT Filed: Jun. 1, 2023
(86) PCT No.: PCT/US2023/067771

§ 371 Date: Nov. 26, 2024

Related U.S. Application Data
(60) Provisional application No. 63/347,665, filed on Jun. 1, 2022.

(51) Int. Cl. A61K 31/711; A61P 35/00; C07K 16/28; C12N 15/11; C12N 15/63

(52) U.S. Cl. CPC A61K 31/711; A61P 35/00; C07K 16/2809; C07K 16/2863; C12N 15/11; C12N 15/63; C07K 2317/31

(57) ABSTRACT

Disclosed herein are compositions comprising a recombinant nucleic acid sequence encoding bispecific T cell engaging (DBTE) antibodies targeting EGFRvIII and HER2 and methods of use of the same for treating glioblastoma.

### CROSS REFERENCE TO RELATED APPLICATIONS

This application claims priority to U.S. Provisional Application No. 63/347,665, filed Jun. 1, 2022 which is hereby incorporated by reference herein in its entirety.

### TECHNICAL FIELD

The present invention relates to compositions comprising a combination of recombinant nucleic acid sequences for generating synthetic DNA encoded bispecific T cell engagers (DBTE), and functional fragments thereof, in vivo, and methods of preventing and/or treating cancer in a subject by administering said compositions.

### BACKGROUND

Glioblastoma multiforme (GBM) is the most lethal and aggressive glioma in adults with a five-year survival rate of less than 5% (Taylor et al., 2019, Front. Oncol. 9, 963). With a standard of care which is comprised of surgical resection, radiation and chemotherapy, the median survival remains 15 months for GBM patients. Approximately 40% of the patients have unresectable GBM and show poorer prognosis due to high recurrence rate (Bausart et al., 2022, J. Exp. Clin. Cancer Res. 41, 35). Currently, there is no Food and Drug Administration (FDA)-approved immunotherapy for GBM patients. The poor prognosis and the lack of alternative therapy illustrate the highly unmet clinical need of new therapies for GBM patients.

Recently, immunotherapies targeting epidermal growth factor receptor (EGFR) variant III (EGFRvIII) are receiving attention as potential treatment options for GBM. Epidermal growth factor receptor (EGFR) variant III (EGFRvIII) is the most frequent mutant form of EGFR which results from in-frame deletion of the EGF ligand-binding domain (Felsberg et al., 2017, Clin. Cancer Res. 23, 6846-6855). EGFRvIII is an oncogenic, tumor-specific surface antigen that is present on up to 30% of newly diagnosed GBM cases and is undetectable in normal tissues, making it an ideal target for immunotherapy (Felsberg et al., 2017, Clin. Cancer Res. 23, 6846-6855; Padfield et al., 2015, Front. Oncol. 5, 5). Immunotherapies targeting EGFRvIII are receiving attention as potential treatment options for GBM. These EGFRvIII-targeted approaches previously tested in clinical trials include chimeric antigen receptor T cells (CAR-T) as well as studies with a peptide vaccine strategy (O'Rourke et al., 2017, Sci. Transl. Med. 9, eaaa0984; Schuster et al., 2015, Neuro. Oncol. 17, 854-861). However, they so far have not demonstrated significant survival benefits beyond the standard of care, with one obstacle reported of targeted antigen loss, resulting in tumor escape in treated patients.

Immune escape poses a significant challenge for antigen-targeted immunotherapies for GBM which manifests heterogeneous antigen landscape. GBM exhibits various degrees of antigenic heterogeneity. Clinical studies revealed that the expressions of antigens such as EGFRvIII and HER2 were highly heterogeneous in GBM patient samples (Liu et al., 2004, Cancer Res. 64, 4980-4986; Saikali et al., 2007, J. Neurooncol. 81, 139-148). The antigen heterogeneity could be driven in part from tumor cells that evade immune surveillance by downregulation, mutation, deletion of antigen, and selective survival of antigen-negative tumor subpopulations (Nagaraj et al., 2007, Nat. Med. 13, 828-835; Funari et al., 2015, Nat. Rev. Cancer 15, 302-310; Vinay et al., 2015, Semin. Cancer Biol. 35, S185-S198). Such mechanisms of antigen escape create challenges for single antigen-targeted approaches in effectively eliminating the entire tumor burden and preventing recurrence. Thus, strategies that can target multiple tumor antigens simultaneously may be of importance for GBM patients.

Bispecific T cell engagers (BTEs) are bispecific antibodies that induce T cell-mediated anti-tumor cytotoxicity and have demonstrated promising results in targeting solid tumors in preclinical studies (Goebeler et al., 2020, Nat. Rev. Clin. Oncol. 17, 418-434; Zhou et al., 2021, Biomark. Res. 9, 38). An EGFRvIII-targeting BTE was studied in an animal model of GBM, which demonstrated moderate tumor control as well as survival through delivery of 16 consecutive daily doses (Stemjak et al., 2021, Mol. Cancer Ther. 20, 925-933). Improving potency and in vivo pharmacokinetics are important for further development. Direct in vivo delivery of BTEs with more durable expression remains an important goal for study in therapeutic models of GBM. Such an approach could simplify clinical translation, providing patient benefit by improved pharmacokinetics likely with lower costs.

There remains a need in the art for longer-lived, simpler production, antibody-based products for cancer immunotherapy. The current invention satisfies this need.

### SUMMARY OF THE INVENTION

In one embodiment, the invention relates to a nucleic acid molecule encoding one or more synthetic DNA encoded bispecific immune cell engager, wherein the more synthetic DNA encoded bispecific immune cell engager comprises at least one least one antigen binding domain, and at least one immune cell engaging domain. In one embodiment, the antigen binding domain targets epidermal growth factor receptor variant III (EGFRvIII), human epidermal growth factor receptor 2 (Her2), or a combination thereof.

In one embodiment, the immune cell engaging domain targets a T cell, an antigen presenting cell, a natural killer (NK) cell, a neutrophil or a macrophage. In one embodiment, the immune cell engaging domain targets CD3, the T cell receptor (TCR), CD28, CD16, NKG2D, Ox40, 4-1BB, CD2, CD5, CD40, FcgRs, FceRs, FcaRs or CD95. In one embodiment, the immune cell engaging domain targets CD3.

In one embodiment, the nucleic acid molecule comprises a nucleotide sequence encoding an amino acid sequence having at least about 90% identity over an entire length of the amino acid sequence to SEQ ID NO:2, SEQ ID NO:4, SEQ ID NO:6, or SEQ ID NO:8. In one embodiment, the nucleic acid molecule comprises a nucleotide sequence encoding a fragment of an amino acid sequence having at least about 90% identity over at least 65% of the amino acid sequence to SEQ ID NO:2, SEQ ID NO:4, SEQ ID NO:6, or SEQ ID NO:8. In one embodiment, the nucleic acid molecule comprises a nucleotide sequence encoding an amino acid sequence of SEQ ID NO:2, SEQ ID NO:4, SEQ ID NO:6, or SEQ ID NO:8. In one embodiment, the nucleic acid molecule comprises a nucleotide sequence encoding a fragment of an amino acid sequence comprising at least 65% of an amino acid sequence of SEQ ID NO:2, SEQ ID NO:4, SEQ ID NO:6, or SEQ ID NO:8.

In one embodiment, the nucleic acid molecule comprises a nucleotide sequence having at least about 90% identity over an entire length of the nucleic acid sequence to SEQ ID NO: 1, SEQ ID NO:3, SEQ ID NO:5, or SEQ ID NO:7. In one embodiment, the nucleic acid molecule comprises a fragment of a nucleotide sequence having at least about 90% identity over at least 65% of SEQ ID NO:1, SEQ ID NO:3, SEQ ID NO:5, or SEQ ID NO:7. In one embodiment, the nucleic acid molecule comprises a nucleotide sequence of SEQ ID NO:1, SEQ ID NO:3, SEQ ID NO:5, or SEQ ID NO:7. In one embodiment, the nucleic acid molecule comprises a fragment of a nucleotide sequence comprising at least 65% of a nucleotide sequence of SEQ ID NO:1, SEQ ID NO:3, SEQ ID NO:5, or SEQ ID NO:7.

In one embodiment, the nucleotide sequence is operably linked to a nucleic acid sequence encoding an IgE leader sequence.

In one embodiment, the nucleic acid molecule comprises an expression vector.

In one embodiment, the invention relates to a composition comprising at least one nucleic acid molecule encoding one or more synthetic DNA encoded bispecific immune cell engager, wherein the more synthetic DNA encoded bispecific immune cell engager comprises at least one least one antigen binding domain, and at least one immune cell engaging domain. In one embodiment, the antigen binding domain targets epidermal growth factor receptor variant III (EGFRvIII), human epidermal growth factor receptor 2 (Her2), or a combination thereof. In one embodiment, the composition further comprises a pharmaceutically acceptable excipient.

In one embodiment, the composition comprises a first synthetic DNA encoded bispecific immune cell engager comprising an antigen binding domain targeting EGFRvIII and a second synthetic DNA encoded bispecific immune cell engager comprising an antigen binding domain targeting HER2. In one embodiment, the immune cell engaging domain for each of the first and second synthetic DNA encoded bispecific immune cell engager targets a T cell, an antigen presenting cell, a natural killer (NK) cell, a neutrophil or a macrophage. In one embodiment, the immune cell engaging domain for each of the first and second synthetic DNA encoded bispecific immune cell engager targets CD3, the T cell receptor (TCR), CD28, CD16, NKG2D, Ox40, 4-1BB, CD2, CD5, CD40, FcgRs, FceRs, FcaRs or CD95. In one embodiment, the immune cell engaging domain for each of the first and second synthetic DNA encoded bispecific immune cell engager targets CD3.

In one embodiment, the first synthetic DNA encoded bispecific immune cell engager comprising an antigen binding domain targeting EGFRvIII comprises a nucleotide sequence encoding an amino acid sequence having at least about 90% identity over an entire length of the amino acid sequence to SEQ ID NO:2 or SEQ ID NO:4.

In one embodiment, the first synthetic DNA encoded bispecific immune cell engager comprising an antigen binding domain targeting EGFRvIII comprises a fragment of an amino acid sequence having at least about 90% identity over at least 65% of the amino acid sequence to SEQ ID NO:2 or SEQ ID NO:4. In one embodiment, the first synthetic DNA encoded bispecific immune cell engager comprising an antigen binding domain targeting EGFRvIII comprises an amino acid sequence of SEQ ID NO:2 or SEQ ID NO:4. In one embodiment, the first synthetic DNA encoded bispecific immune cell engager comprising an antigen binding domain targeting EGFRvIII comprises a fragment of an amino acid sequence comprising at least 65% of an amino acid sequence of SEQ ID NO:2 or SEQ ID NO:4.

In one embodiment, the second synthetic DNA encoded bispecific immune cell engager comprising an antigen binding domain targeting HER2 comprises a nucleotide sequence encoding an amino acid sequence having at least about 90% identity over an entire length of the amino acid sequence of SEQ ID NO:6 or SEQ ID NO:8. In one embodiment, the second synthetic DNA encoded bispecific immune cell engager comprising an antigen binding domain targeting HER2 comprises a fragment of an amino acid sequence having at least about 90% identity over at least 65% of the amino acid sequence to an amino acid sequence of SEQ ID NO:6 or SEQ ID NO:8. In one embodiment, the second synthetic DNA encoded bispecific immune cell engager comprising an antigen binding domain targeting HER2 comprises an amino acid sequence of SEQ ID NO:6 or SEQ ID NO:8. In one embodiment, the second synthetic DNA encoded bispecific immune cell engager comprising an antigen binding domain targeting HER2 comprises a fragment of an amino acid sequence comprising at least 65% of an amino acid sequence of SEQ ID NO:6 or SEQ ID NO:8.

In one embodiment, the first synthetic DNA encoded bispecific immune cell engager comprising an antigen binding domain targeting EGFRvIII comprises a nucleotide sequence having at least about 90% identity over an entire length of the nucleic acid sequence to SEQ ID NO:1 or SEQ ID NO:3. In one embodiment, the first synthetic DNA encoded bispecific immune cell engager comprising an antigen binding domain targeting EGFRvIII comprises a fragment of a nucleotide sequence having at least about 90% identity over at least 65% of the nucleic acid sequence to a nucleotide sequence of SEQ ID NO:1 or SEQ ID NO:3. In one embodiment, the first synthetic DNA encoded bispecific immune cell engager comprising an antigen binding domain targeting EGFRvIII comprises a nucleotide sequence of SEQ ID NO:1 or SEQ ID NO:3. In one embodiment, the first synthetic DNA encoded bispecific immune cell engager comprising an antigen binding domain targeting EGFRvIII comprises a fragment of a nucleotide sequence comprising at least 65% of a nucleotide sequence of SEQ ID NO:1 or SEQ ID NO:3.

In one embodiment, the second synthetic DNA encoded bispecific immune cell engager comprising an antigen binding domain targeting HER2 comprises a nucleotide sequence having at least about 90% identity over an entire length of the nucleic acid sequence to a nucleotide sequence of SEQ ID NO:5 or SEQ ID NO:7. In one embodiment, the second synthetic DNA encoded bispecific immune cell engager comprising an antigen binding domain targeting HER2 comprises a fragment of a nucleotide sequence having at least about 90% identity over at least 65% of the nucleic acid sequence to a nucleotide sequence of SEQ ID NO:5 or SEQ ID NO:7. In one embodiment, the second synthetic DNA encoded bispecific immune cell engager comprising an antigen binding domain targeting HER2 comprises a nucleotide sequence of SEQ ID NO:5 or SEQ ID NO:7. In one embodiment, the second synthetic DNA encoded bispecific immune cell engager comprising an antigen binding domain targeting HER2 comprises a fragment of a nucleotide sequence comprising at least 65% of a nucleotide sequence of SEQ ID NO:5 or SEQ ID NO:7.

In one embodiment, the invention relates to a method of preventing or treating a disease or disorder in a subject, the method comprising administering to the subject a nucleic acid molecule encoding one or more synthetic DNA encoded bispecific immune cell engager, wherein the more synthetic DNA encoded bispecific immune cell engager comprises at least one least one antigen binding domain, and at least one immune cell engaging domain, or a composition comprising a combination of nucleic acid molecules encoding one or more synthetic DNA encoded bispecific immune cell engager, wherein the more synthetic DNA encoded bispecific immune cell engager comprises at least one least one antigen binding domain, and at least one immune cell engaging domain.

In one embodiment, the disease is a benign tumor, cancer or a cancer-associated disease. In one embodiment, the disease is glioblastoma.

### BRIEF DESCRIPTION OF THE DRAWINGS

FIG. 1A-1E: Design, in vitro expression, and binding assessment of EGFRvIII-DBTE. (FIG. 1A) Design and structure of EGFRvIII-targeted DBTE. (FIG. 1B) Western blot of supernatant of Expi293F cells transfected with EGFRvIII-DBTE or pVAX (vehicle control). (FIG. 1C) EGFRvIII-DBTE on-cell binding to EGFRvIII (target cells) and CD3 (effector cells) was evaluated by flow cytometry. (FIG. 1D) Flow cytometry data showing the effector cells binding to the target cells in the presence of EGFRvIII-DBTE. The flow panels were gated on U87 cells. GFP is a reporter of EGFRvIII on U87 cells. (FIG. 1E) Fluorescent images showing T cells clustering around the U87vIII cells after 5 hours of incubation.

FIG. 2A-2E: T cell-mediated Cytotoxicity of EGFRvIII-DBTE. EGFRvIII-DBTE was added (10 ng/ml) to the U87vIII cells (10,000 cells/well) and their viabilities were measured in real-time by xCelligence RTCA for 48 hours (FIG. 2A) with human T cells, (FIG. 2B) without human T cells, and (FIG. 2C) with human T cells in the absence of EGFRvIII on tumor cells (U87-MG). (FIG. 2D) % cytolysis data at 48 hour with T cells from 4 different donors were plotted to determine EC50 value. E:T ratio was 10:1 for FIG. 2A-2D. (FIG. 2E) % cytolysis of EGFRvIII-DBTE against U87vIII in various effector to target ratios.

FIG. 3A-3G: EGFRvIII-DBTE induces T cell activation. (FIG. 3A) Fluorescent images of U87vIII cells in a tumor-killing assay upon addition of mouse sera treated with EGFRvIII-DBTE or pVAX1. Day 14 sera were used. Target cells are shown in green. CD69 activation is shown in red. Caspase-3 induction is shown in blue. (FIG. 3B-3D) Quantified GFP+ cell counts, CD69 activation, and caspase-3 induction in the tumor-killing assay. Flow cytometry data showing (FIG. 3E) IFN-g, TNF-α, IL-2, and (FIG. 3F) CD107a responses in CD4+ T cells and CD8+ T cells in a 24-h tumor-killing assay. (FIG. 3G) Tumor-killing assay with CD4+ T cells and/or CD8+ T cells in the presence of EGFRvIII-DBTE. (FIG. 3H) Fluorescent images of the target cells at 0-, 6- and 24-h time points.

FIG. 4A-4B: In vivo expression of EGFRvIII-DBTE. (FIG. 4A) T cell-mediated cytotoxicity against U87vIII induced by sera of NSG treated with a single injection (100 μg) of pVAX, EGFRvIII-DBTE, or recombinant EGFRvIII-DBTE. Sera were collected from day 0 to 105 (FIG. 4B) Day 14 sera of various doses of EGFRvIII-DBTE induced T cell-mediated cytotoxicity against U87vIII cells. % cytolysis data after 48-hour incubation were plotted for FIG. 4A and FIG. 4B.

FIG. 5A-5B: Heterotopic GBM challenge. (FIG. 5A) The scheme of heterotopic GBM challenge study in NSG mice. (n=5) (FIG. 5B) Tumor volume measured by digital caliper in the challenge study plotted over time. (FIG. 5C) IVIS images of tumors in the flank of NSG mice with bioluminescent signals.

FIG. 6A-6E: Intracerebral GBM challenge. (FIG. 6A) A scheme of intracranial GBM challenge in NSG mice. (FIG. 6B) Tumor burden of the challenged mice measured by IVIS. (FIG. 6C) IVIS images of the challenged mice. (FIG. 6D) Survival of the challenged mice. (FIG. 6E) Representative confocal images of brain sections of the challenged NSG mice at the endpoint of the study. EGFRvIII expression is shown in magenta. Nuclei are shown in yellow.

FIG. 7A-7B: EGFRvIII+/HER2+ heterogeneous model of GBM. (FIG. 7A) T cell-mediated cytotoxicity assay against U87vIII cells (EGFRvIII+) and/or U251 cells (HER2+) using NSG mice treated with EGFRvIII-DBTE and/or HER2-DBTE. (FIG. 7B) Fluorescent images of the heterogeneous tumor mixture (U87vIII/U251) in a 48-h tumor-killing assay.

FIG. 8A-8H: Co-delivery of EGFRvIII-DBTE and HER2-DBTE in heterogeneous GBM challenge. (FIG. 8A) A scheme of heterogeneous orthotopic GBM challenge in NSG mice wherein a mixture of U87vIII cells and U251 cells were inoculated in the brain. (FIG. 8B-8E) Tumor burden of the challenged mice that received a treatment of (FIG. 8B) pVAX1, (FIG. 8C) EGFRvIII-DBTE, (FIG. 8D) HER2-DBTE, or (FIG. 8E) both EGFRvIII-DBTE and HER2-DBTE. (FIG. 8F) Survival of the challenged NSG mice. (FIG. 8G) IVIS images of the challenged mice. (FIG. 8H) Representative confocal images of the brain sections of the challenged mice at the endpoints of the study. EGFRvIII expression is shown in magenta. HER2 expression is shown in cyan. Nuclei are shown in yellow.

FIG. 9A-9B: GBM cell lines and antigen expression. (FIG. 9A) Flow cytometry data and uorescent images showing EGFRvIII expression in wildtype U87 cells and U87vIII cells. (FIG. 9B) Flow cytometry data showing EGFRvIII and HER2 expression in U87vIII cells and U251 cells.

FIG. 10A-10C: Binding ELISA of EGFRvIII-DBTE. Binding ELISA of EGFRvIII-DBTE (transfection supernatant) against recombinant (FIG. 10A) EGFRvIII, (FIG. 10B) CD3e, and (FIG. 10C) wildtype EGFR.

### DETAILED DESCRIPTION

The present invention relates to compositions comprising a recombinant nucleic acid sequence encoding a bispecific immune cell engaging antibody (DICE), a recombinant nucleic acid sequence encoding a bispecific T cell engaging (DBTE) antibody, a fragment thereof, a variant thereof, or a combination thereof. The composition can be administered to a subject in need thereof to facilitate in vivo expression and formation of a DBTE.

In one embodiment, the DBTE comprises at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the antigen binding domain comprises a nucleotide sequence encoding an antibody, a fragment thereof, or a variant thereof specific for binding to an antigen. In one embodiment, the antibody or fragment thereof is a DNA encoded bispecific T cell engaging binding molecule (DBTE) or a fragment or variant thereof.

In one embodiment, the antigen binding domain of the DBTE is specific for binding a target antigen, and recruiting a T cell to the target antigen. In one embodiment, the target antigen is a tumor antigen. In one embodiment, the antigen is epidermal growth factor receptor variant III (EGFRvIII), or human epidermal growth factor receptor 2 (Her2). Therefore, in one embodiment, the invention provides compositions comprising one or more DBTE and methods for use in treating or preventing cancer or a disease or disorder associated with cancer in a subject.

In one embodiment, the invention provides a combination of DBTEs, wherein the combination of DBTES comprises a first DBTE comprising at least one antigen binding domain, and at least one immune cell engaging domain and a second DBTE comprising at least one antigen binding domain, and at least one immune cell engaging domain. In one embodiment, the immune cell engaging domain of each DBTE is specific for an antigen expressed on the surface of an immune cell.

In one embodiment, the combination of DBTE targets EGFRvIII, and Her2. Therefore, in one embodiment, the invention provides compositions comprising a combination of an EGFRvIII DBTE and a HER2 DBTE and methods for use in treating or preventing cancer or a disease or disorder associated with cancer in a subject. In some embodiments, the cancer is glioblastoma.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or anon-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering an antibody of the present invention to a subject prior to onset of the disease. Suppressing the disease involves administering a antibody of the present invention to a subject after induction of the disease but before its clinical appearance. Repressing the disease involves administering an antibody of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Compositions

In one embodiment, the present invention relates to compositions comprising a recombinant nucleic acid sequence encoding a DBTE, a fragment thereof, a variant thereof, or a combination thereof. The compositions, when administered to a subject in need thereof, can result in the generation of a synthetic DNA encoded bispecific immune cell engager in the subject.

In one embodiment, the DBTE comprisies at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the invention relates to a combination of nucleic acid-encoded immune cell engaging antibodies of the invention. In various embodiments, the combination of nucleic acid-encoded immune cell engaging antibodies of the invention target two or more different tumor antigens. In one embodiment, the two or more tumor antigens are epidermal growth factor receptor variant III (EGFRvIII) and human epidermal growth factor receptor 2 (Her2).

In one embodiment, the invention relates to compositions comprising a combination of two or more bispecific T cell engaging antibodies. In some embodiments, the two or more bispecific T cell engaging antibodies of the invention both target the same T cell receptor. For example, in one embodiment, the invention relates to administration of a combination of a first EGFRvIII-CD3 bispecific antibody and a second HER2-CD3 bispecific antibody. In some embodiments, one or more of the combination of bispecific T cell engaging antibodies of the invention comprises a mutation in the Fc domain to promote heterodimerization and increase the serum half-life of the antibody.

In one embodiment, a nucleotide sequence encoding a EGFRvIII DBTE encodes the amino acid sequence of SEQ ID NO:2 or SEQ ID NO:4 or a fragment or variant thereof. In one embodiment, a nucleotide sequence encoding a EGFRvIII DBTE comprises a nucleotide sequence of SEQ ID NO:1 or SEQ ID NO:3 or a fragment or variant thereof.

In one embodiment, a nucleotide sequence encoding a HER2 DBTE encodes the amino acid sequence of SEQ ID NO:6 or SEQ ID NO:8 or a fragment or variant thereof. In one embodiment, a nucleotide sequence encoding a HER2 DBiTE comprises a nucleotide sequence of SEQ ID NO:5 or SEQ ID NO:7 or a fragment or variant thereof.

In one embodiment, a nucleotide sequence encoding a EGFRvIII DBTE encodes at least one amino acid sequence of SEQ ID NO:2 or SEQ ID NO:4, or a fragment or variant thereof. In one embodiment, the fragment of SEQ ID NO:2 or SEQ ID NO:4 is a binding fragment comprising at least one, at least two, at least three, at least four, at least five, at least six, at least seven, at least 8, at least 9, at least 10, at least 11 or all 12 CDR sequences of SEQ ID NO:2 or SEQ ID NO:4. In some embodiments, the binding fragment comprises at least three CDR sequences of SEQ ID NO:2 or SEQ ID NO:4. In one embodiment, a nucleotide sequence encoding a EGFRvIII DBTE comprises at least one nucleotide sequence of SEQ ID NO:1 or SEQ ID NO:3 or a fragment or variant thereof. In one embodiment, the fragment of SEQ ID NO:1 or SEQ ID NO:3 encodes a binding fragment of a DBTE of the invention, and comprises at least one, at least two, at least three, at least four, at least five, at least six, at least seven, at least eight, at least nine, at least ten, at least eleven or all twelve CDR coding sequences of EGFRvIII DBTE, encoding at least one, at least two, at least three, at least four, at least five, at least six, at least seven, at least eight, at least nine, at least ten, at least eleven or all twelve CDR sequences of SEQ ID NO:2 or SEQ ID NO:4. In some embodiments, the fragment of SEQ ID NO:1 or SEQ ID NO:3 encodes a binding fragment of a DBTE of the invention comprising at least three CDR sequences of SEQ ID NO:2 or SEQ ID NO:4. In some embodiments, the fragment of SEQ ID NO:1 or SEQ ID NO:3 encodes a binding fragment of a DBTE of the invention comprising at least six CDR sequences of SEQ ID NO:2 or SEQ ID NO:4.

In one embodiment, a nucleotide sequence encoding a HER2 DBTE encodes at least one amino acid sequence of SEQ ID NO:6 or SEQ ID NO:8, or a fragment or variant thereof. In one embodiment, the fragment of SEQ ID NO:6 or SEQ ID NO:8 is a binding fragment comprising at least one, at least two, at least three, at least four, at least five, at least six, at least seven, at least 8, at least 9, at least 10, at least 11 or all 12 CDR sequences of SEQ ID NO:6 or SEQ ID NO:8. In some embodiments, the binding fragment comprises at least three CDR sequences of SEQ ID NO:6 or SEQ ID NO:8. In one embodiment, a nucleotide sequence encoding a HER2 DBTE comprises at least one nucleotide sequence of SEQ ID NO:5 or SEQ ID NO:7 or a fragment or variant thereof. In one embodiment, the fragment of SEQ ID NO:5 or SEQ ID NO:7 encodes a binding fragment of a DBTE of the invention, and comprises at least one, at least two, at least three, at least four, at least five, at least six, at least seven, at least eight, at least nine, at least ten, at least eleven or all twelve CDR coding sequences of HER2 DBTE, encoding at least one, at least two, at least three, at least four, at least five, at least six, at least seven, at least eight, at least nine, at least ten, at least eleven or all twelve CDR sequences of SEQ ID NO:6 or SEQ ID NO:8. In some embodiments, the fragment of SEQ ID NO:5 or SEQ ID NO:7 encodes a binding fragment of a DBTE of the invention comprising at least three CDR sequences of SEQ ID NO:6 or SEQ ID NO:8. In some embodiments, the fragment of SEQ ID NO:5 or SEQ ID NO:7 encodes a binding fragment of a DBTE of the invention comprising at least six CDR sequences of SEQ ID NO:6 or SEQ ID NO:8.

In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined amino acid sequence. In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of one or more of SEQ ID NO: 2, 4, 6, or 8.

A binding arm of a bispecific T cell engaging antibody, or antigen-binding fragment thereof, may include, but is not limited to a polyclonal antibody, a monoclonal fusion proteins, antibodies or fragments thereof, chimerized or chimeric fusion proteins, antibodies or fragments thereof, humanized fusion proteins, antibodies or fragments thereof, deimmunized humfusion proteins, antibodies or fragments thereof, fully humfusion proteins, antibodies or fragments thereof, single chain antibody, single chain Fv fragment (scFv), Fv, Fd fragment, Fab fragment, Fab′ fragment, F(ab′)₂ fragment, diabody or antigen-binding fragment thereof, minibody or antigen-binding fragment thereof, triabody or antigen-binding fragment thereof, domain fusion proteins, antibodies or fragments thereof, camelid fusion proteins, antibodies or fragments thereof, dromedary fusion proteins, antibodies or fragments thereof, phage-displayed fusion proteins, antibodies or fragments thereof, or antibody, or antigen-binding fragment thereof, identified with a repetitive backbone array (e.g. repetitive antigen display).

In certain embodiments, the composition can treat, prevent, and or/protect against a disease or disorder associated with the antigen to which the synthetic antibody (e.g., DMAb, ScFv antibody fragment, DBTE) of the invention binds. In one embodiment, the composition of the invention can treat, prevent and/or protect against any disease, disorder, or condition associated with expression of the targeted antigen. In certain embodiments, the composition can treat, prevent, and or/protect against cancer.

The synthetic antibody (e.g., DMAb, ScFv antibody fragment, DBTE) can treat, prevent, and/or protect against disease in the subject administered the composition. The synthetic antibody (e.g., DMAb, ScFv antibody fragment, DBTE) can promote survival of the disease in the subject administered the composition. The synthetic antibody (e.g., DMAb, ScFv antibody fragment, DBTE) can provide at least about 50%, 55%, 60%, 65%, 70%, 75%, 80%, 85%, 90%, 95%, or 100% survival of the disease in the subject administered the composition. In other embodiments, the synthetic antibody (e.g., DMAb, ScFv antibody fragment, DBTE) can provide at least about 65%, 66%, 67%, 68%, 69%, 70%, 71%, 72%, 73%, 74%, 75%, 76%, 77%, 78%, 79%, or 80% survival of the disease in the subject administered the composition. In some embodiments, the combination of synthetic antibodies (e.g., EGFRvIII and HER2 DBTEs) can provide at least about 65%, 66%, 67%, 68%, 69%, 70%, 71%, 72%, 73%, 74%, 75%, 76%, 77%, 78%, 79%, or 80% survival of the disease in the subject administered the composition.

The composition can result in the generation of the synthetic antibody (e.g., DMAb, ScFv antibody fragment, DBTE) in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody (e.g., DMAb, ScFv antibody fragment, DBTE) in the subject within at least about 1 day, 2 days, 3 days, 4 days, 5 days, 6 days, 7 days, 8 days, 9 days, or 10 days of administration of the composition to the subject. The composition can result in generation of the synthetic antibody (e.g., DMAb, ScFv antibody fragment, DBTE) in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody (e.g., DMAb, ScFv antibody fragment, DBTE) in the subject more quickly than the generation of an endogenous antibody in a subject who is administered an antigen to induce a humoral immune response. The composition can result in the generation of the synthetic antibody (e.g., DMAb, ScFv antibody fragment, DBTE) at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### Recombinant Nucleic Acid Sequence

As described above, the composition can comprise a recombinant nucleic acid sequence. The recombinant nucleic acid sequence can encode the synthetic antibody (e.g., DMAb, ScFv antibody fragment, DBTE), a fragment thereof, a variant thereof, or a combination thereof. The antibody is described in more detail below.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### Heavy Chain Polypeptide

The recombinant nucleic acid sequence construct can include a heterologous nucleic acid encoding a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### Light Chain Polypeptide

The recombinant nucleic acid sequence construct can include a heterologous nucleic acid sequence encoding a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides. In one embodiment, the linker sequence is a G4S linker sequence, having an amino acid sequence of GGGGSGGGGSGGGGS (SEQ ID NO:9).

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody (e.g., DMAb, ScFv antibody fragment, DBTE). In particular, the heavy chain polypeptide and the light chain polypeptide can interact with one another such that assembly results in the synthetic antibody (e.g., DMAb, ScFv antibody fragment, DBTE) being capable of binding the antigen. In other embodiments, the heavy chain polypeptide and the light chain polypeptide can interact with one another such that assembly results in the synthetic antibody (e.g., DMAb, ScFv antibody fragment, DBTE) being more immunogenic as compared to an antibody not assembled as described herein. In still other embodiments, the heavy chain polypeptide and the light chain polypeptide can interact with one another such that assembly results in the synthetic antibody (e.g., DMAb, ScFv antibody fragment, DBTE) being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### RNA

In one embodiment, the nucleic acid is an RNA molecule. In one embodiment, the RNA molecule is transcribed from a DNA sequence. Accordingly, in one embodiment, the invention provides an RNA molecule encoding one or more of the synthetic antibodies of the invention. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### Antibody

In some embodiments, the invention relates to a recombinant nucleic acid sequence encoding an antibody, a fragment thereof, a variant thereof, or a combination thereof. The antibody can bind or react with an antigen, which is described in more detail below. In some embodiments, the antibody is a DNA encoded monoclonal antibody (DMAb), a fragment thereof, or a variant thereof. In some emboidments the fragment is an ScFv fragment. In some embodiments, the antibody is a DNA encoded bispecific T cell engagers (BTE), a fragment thereof, or a variant thereof.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

Bispecific T cell Engager

As described above, the recombinant nucleic acid sequence can encode a bispecific T cell engager (BTE), a fragment thereof, a variant thereof, or a combination thereof. The antigen targeting domain of the BTE can bind or react with the antigen, which is described in more detail below.

The antigen targeting domain of the BTE may comprise an antibody, a fragment thereof, a variant thereof, or a combination thereof. The antigen targeting domain of the BTE may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding domain, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antigen targeting domain of the BiTE can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antigen targeting domain of the BiTE can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antigen targeting domain of the BiTE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

In one embodiment, at least one of the antigen binding domaining and the immune cell engaging domain of the DBTE of the invention is a ScFv DNA encoded monoclonal antibody (ScFv DMAb) as described in detail above.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments, one of the binding sites is capable of an tumor antigen. In some embodiments, the binding site included in the Fab fragment is a binding site specific for a tumor antigen. In some embodiments, the binding site included in the single chain Fv fragment is a binding site specific for a tumor antigen such as EGFR or Her2.

In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing .alpha.- and .beta.-chains, in some embodiments, it encompasses .gamma.-chains and .delta.-chains (supra). Accordingly, in some embodiments, the TCR is TCR (alpha/beta) and in some embodiments, it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments, a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds a tumor antigen and the second binding site binds a T cell specific receptor molecule and/or a natural killer (NK) cell specific receptor molecule.

In some embodiments, the first binding site of the antibody molecule binds EGFR or Her2, and the second binding site binds a T cell specific receptor molecule and/or a natural killer (NK) cell specific receptor molecule. In some embodiments, the first binding site of the antibody molecule binds EGFR or Her2 and the second binding site binds one of CD3, TCR, CD28, CD16, NKG2D, Ox40, 4-1BB, CD2, CD5, CD40, FcgRs, FceRs, FcaRs and CD95. In some embodiments, the first binding site of the antibody molecule binds EGFR or Her2 and the second binding site binds CD3.

In some embodiments, the first binding site of the antibody molecule binds a T cell specific receptor molecule and/or a natural killer (NK) cell specific receptor molecule and the second binding site binds a tumor antigen. In some embodiments, the first binding site of the antibody binds a T cell specific receptor molecule and/or a natural killer (NK) cell specific receptor molecule and the second binding site binds EGFR or Her2. In some embodiments, the first binding site of the antibody binds one of CD3, TCR, CD28, CD16, NKG2D, Ox40, 4-1BB, CD2, CD5, CD40, FcgRs, FceRs, FcaRs and CD95, and the second binding site binds EGFR or Her2. In some embodiments, the first binding site of the antibody binds CD3, and the second binding site binds EGFR or Her2.

In one embodiment the bispecific antibody of the invention comprises a DBTE, comprising one or more scFv antibody fragments as described herein, thereby allowing the DBTE to bind or react with the desired target molecules.

In one embodiment the DBTE, comprises a nucleic acid molecule encoding a first scFv specific for binding to a target disease-specific antigen linked to a second scFv specific for binding to a T cell specific receptor molecule. The linkage may place the first and second domains in any order, for example, in one embodiment, a nucleotide sequence encoding a scFv specific for binding to a target disease-specific antigen is oriented 5′ (or upstream) to a nucleotide sequence encoding a scFv specific for binding to a T cell specific receptor molecule. In another embodiment, a nucleotide sequence encoding a scFv specific for binding to a target disease-specific antigen is oriented 3′ (or downstream) to a nucleotide sequence encoding a scFv specific for binding to a T cell specific receptor molecule.

In some embodiments, the invention provides a combination of bispecific binding molecules, wherein each bispecific binding molecule comprises a first binding site that binds to a tumor antigen and a second binding site that binds to a T cell specific receptor molecule and/or a natural killer (NK) cell specific receptor molecule. In some embodiments, the combination of bispecific binding molecules comprises a first bispecific binding molecule comprising a first binding site specific for EGFR and a second binding site specific for CD3, and a second bispecific binding molecule comprising a first binding site specific for HER2 and a second binding site specific for CD3.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

As described above, the synthetic antibody (e.g., DMAb, ScFv antibody fragment, DBTE) may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### Antigen

In one embodiment, the synthetic antibody (e.g., DMAb, ScFv antibody fragment, DBTE) is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be a tumor antigen. The antigen can be associated with increased risk of cancer development or progression. In one embodiment, the invention provides a combination of bispecific binding molecules specific for binding two or more tumor antigens. In some embodiments, the combination of binding molecules are specific for the combination of EGFR and Her2.

In one embodiment, the invention provides a combination of synthetic nucleic acid encoded bispecific immune cell engagers targeting two or more antigens, wherein each of the synthetic nucleic acid encoded bispecific immune cell engagers further targets a T-cell activating antigen.

### Tumor Antigen

The antigen binding domain of the synthetic antibody (e.g., DMAb, ScFv antibody fragment, DBTE) of the invention can interact with a tumor antigen. In the context of the present invention, “tumor antigen” or “hyperproliferative disorder antigen” or “antigen associated with a hyperproliferative disorder,” refers to antigens that are common to specific hyperproliferative disorders such as cancer.

The type of tumor antigen referred to in the invention may be a tumor-specific antigen (TSA) or a tumor-associated antigen (TAA). A TSA is unique to tumor cells and does not occur on other cells in the body. A TAA antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, CD123, B-cell maturation antigen (BCMA), Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2, Her3, IGFR, CD133, IL3R, fibroblast activating protein (FAP), CDCP1, Derlini, Tenascin, frizzled 1-10, the vascular antigens VEGFR2 (KDR/FLK1), VEGFR3 (FLT4, CD309), PDGFR-α (CD140a), PDGFR-.beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRvIII, EpCAM, Ep-CAM, Folate-binding protein, G250, Fms-like tyrosine kinase 3 (FLT-3, CD135), follicle stimulating hormone receptor (FSHR), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

In one embodiment, the tumor antigen is a hormone or fragment thereof which can be used to target a specific receptor. Examples include, but are not limited to, FSH hormone, LH hormone, TSH hormone or fragments thereof.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Aspects of the present invention include compositions for enhancing an immune response against an antigen in a subject in need thereof, comprising a synthetic antibody (e.g., DMAb, ScFv antibody fragment, DBTE) capable of generating an immune response in the subject, or a biologically functional fragment or variant thereof. In some embodiments, the antigen is EGFRvIII or HER2. In some embodiments, the invention provides a combination of DBTE, comprising a DBTE targeting EGFRvIII and a DBTE targeting HER2.

### T Cell Specific Receptor

In one embodiment, the DBTE of the invention comprises targeting domain specific for binding to a T cell specific receptor. T cell specific receptors include, but are not limited to, CD3, TCR, CD28, CD16, NKG2D, Ox40, 4-1BB, CD2, CD5, CD40, FcgRs, FceRs, FcaRs and CD95.

In some embodiments, the antigen is EGFRvIII or HER2. In some embodiments, the invention provides a combination of DBTE, comprising a DBTE targeting EGFRvIII and a DBTE targeting HER2. In some embodiments, bot the EGFRvIII DBTE and the HER2 DBTE target CD3.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Method of Generating the Synthetic Antibody

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### Combination Vaccine

In some embodiments, one or more of the combination of bispecific T cell engaging antibodies of the invention comprises a mutation in the Fc domain to promote heterodimerization and increase the serum half-life of the antibody.

In some embodiments therefore, the invention relates to immunogenic compositions, such as vaccines, comprising a combination of two or more bispecific T cell engaging antibodies of the invention, a fragment thereof, or a variant thereof (e.g., a combination of a first EGFRvIII-CD3 DBTE and a second HER2-CD3 DBTE). In some embodiments, the invention relates to immunogenic compositions, such as vaccines, comprising a combination of two or more nucleic acid molecules, wherein the combination of nucleic acid molecule encodes two or more bispecific T cell engaging antibodies of the invention, fragments thereof, or variants thereof.

The immunogenic composition can be used to increase the immune response against a combination of antigens. In some embodiments, the immunogenic composition can be used to increase the immune response against a combination of tumor antigens (e.g., a combination of EGFRvIII and HER2). The combination vaccine can significantly induce an immune response of a subject administered the vaccine, thereby protecting against or treating cancer or associated pathologies. In some embodiments, the cancer is glioblastoma.

The immunogenic composition can be a DNA vaccine, a peptide vaccine, an LNP vaccine or a combination thereof. The DNA vaccine can include a nucleic acid sequence encoding a combination of an EGFRvIII and HER2 DBTE. The nucleic acid sequence can be DNA, RNA, cDNA, a variant thereof, a fragment thereof, or a combination thereof. The nucleic acid sequence can also include additional sequences that encode linker, leader, or tag sequences that are linked to the tumor antigen by a peptide bond.

The two or more bispecific T cell engaging antibodies or two or more nucleic acid molecule encoding two or more bispecific T cell engaging antibodies may be administered using any suitable method such that a combination of two or more bispecific T cell engaging antibodies are present in the subject. In one embodiment, the method may comprise administration of a first composition comprising a bispecific T cell engaging antibody of the invention, or nucleic acid molecules encoding the same, by any of the methods described in detail elsewhere herein and administration of a second composition comprising a bispecific T cell engaging antibody of the invention, or nucleic acid molecules encoding the same less than 1, less than 2, less than 3, less than 4, less than 5, less than 6, less than 7, less than 8, less than 9 or less than 10 days following administration of the first composition comprising a bispecific T cell engaging antibody of the invention, or nucleic acid molecules encoding the same. In one embodiment, the method may comprise administration of a first composition comprising a bispecific T cell engaging antibody of the invention, or nucleic acid molecules encoding the same, by any of the methods described in detail elsewhere herein and administration of a second composition comprising a bispecific T cell engaging antibody of the invention, or nucleic acid molecules encoding the same more than 1, more than 2, more than 3, more than 4, more than 5, more than 6, more than 7, more than 8, more than 9 or more than 10 days following administration of the first composition comprising a bispecific T cell engaging antibody of the invention, or nucleic acid molecules encoding the same. In one embodiment, the method may comprise administration of two or more compositions comprising two or more bispecific T cell engaging antibodies of the invention, or nucleic acid molecules encoding the same concurrently. In one embodiment, the method may comprise administration of a single composition comprising two or more bispecific T cell engaging antibodies of the invention, or nucleic acid molecules encoding the same.

### Method of Delivery of the Composition

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 ρs, 20 ρs, 10 ρs or 1 ρs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119(e) to U.S. Provisional Application Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### Method of Treatment

Also provided herein is a method of treating, protecting against, and/or preventing disease in a subject in need thereof by generating the synthetic antibody (e.g., DMAb, ScFv fragment or DBiTE) in the subject. The method can include administering the composition to the subject. Administration of the composition to the subject can be done using the method of delivery described above.

In certain embodiments, the invention provides a method of treating protecting against, and/or preventing cancer. In one embodiment, the method treats, protects against, and/or prevents tumor growth. In one embodiment, the method treats, protects against, and/or prevents cancer progression. In one embodiment, the method treats, protects against, and/or prevents cancer metastasis.

In one embodiment, the invention provides methods for preventing growth of benign tumors, such as, but not limited to, uterine fibroids. The methods comprise administering an effective amount of one or more of the compositions of the invention to a subject diagnosed with a benign tumor.

Upon generation of the synthetic antibody (e.g., DMAb, ScFv fragment or DBiTE) in the subject, the synthetic antibody (e.g., DMAb, ScFv fragment or DBiTE) can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### Cancer Therapy

The invention provides methods of treating or preventing cancer, or of treating and preventing growth or metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a composition of the invention. The invention further provides a method of inhibiting metastasis in an individual in need thereof, the method comprising administering to the individual an effective metastasis-inhibiting amount of any one of the compositions described herein.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, the compositions of the invention can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the compositions of the invention to a subject diagnosed with a malignant tumor or cancerous cells or to a subject having a tumor or cancerous cells.

The following are non-limiting examples of cancers that can be treated by the methods and compositions of the invention: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor. In one embodiment, the cancer is glioblastoma.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### Nanoparticle Formulations

In one embodiment, the immunogenic composition of the invention may comprise a nanoparticle, including but not limited to a lipid nanoparticle (LNP), comprising an EGFRvIII or HER2 DBTE of the invention, or a LNP comprising a nucleic acid encoding an EGFRvIII or HER2 DBTE of the invention. In some embodiments, the composition comprises or encodes all or part of an EGFRvIII or HER2 DBTE of the invention, or an immunogenically functional equivalent thereof. In some embodiments, the composition comprises an mRNA molecule that encodes all or part of an EGFRvIII or HER2 DBTE of the invention.

In one embodiment, the LNP comprises or encapsulates an RNA molecule encoding at least one amino acid sequence of SEQ ID NO:2, SEQ ID NO:4, SEQ ID NO:6, or SEQ ID NO:8, or a fragment or variant thereof.

In one embodiment, the invention provides a combination of LNP comprising or encapsulating a combination of RNA molecule encoding SEQ ID NO:2 or SEQ ID NO:4 and SEQ ID NO:6 or SEQ ID NO:8, or fragments or variants thereof. In one embodiment, the invention provides a combination of a first LNP comprising or encapsulating an RNA molecule encoding SEQ ID NO:2 or SEQ ID NO:4 and a second LNP comprising or encapsulating an RNA molecule encoding SEQ ID NO:6 or SEQ ID NO:8.

In one embodiment, the composition further comprises one or more additional immunostimulatory agents. Immunostimulatory agents include, but are not limited to, an additional antigen or antigen binding molecule, an immunomodulator, or an adjuvant.

### Generation of Synthetic Antibodies In Vitro and Ex Vivo

In one embodiment, the synthetic antibody (e.g., DMAb, ScFv fragment or DBiTE) is generated in vitro or ex vivo. For example, in one embodiment, a nucleic acid encoding a synthetic antibody (e.g., DMAb, ScFv fragment or DBiTE) can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Simultaneous In Vivo Delivery of DNA-Encoded Bispecific T Cell Engagers (DBTEs) Effectively Controls Heterogeneous GBM Tumors and Mitigates Antigen Escape

In a preliminary study, a DNA-encoded BTE (DBTE) targeting ovarian cancer in a peripheral challenge model was described (Perales-Puchalt et al., 2019, JCI Insight 4, e126086). Here the data build on the prior work focusing on engineering a new in vivo-produced EGFRvIII-targeting DBTE (EGFRvIII-DBTE) first as a monotherapy for direct in vivo treatment for GBM in both peripheral and orthotopic challenge animal models. It is shown that the in vivo expression of the EGFRvIII-DBTE, specificity, T cell-mediated cytotoxicity, and efficacy in challenge models of GBM. A single injection of EGFRvIII-DBTE exhibited durable in vivo expression and potent tumor regression and clearance in mice.

The study is advanced to describe a delivery of multiple DBTEs as a potential combination therapy for heterogeneous GBM. The GBMassociated antigens, EGFRvIII and HER2, are expressed in up to 30% and 80% of GBM cases, respectively (Ahmed et al., 2010, Clin. Cancer Res. 16, 474-485; Felsberg et al., 2017, Clin. Cancer Res. 23, 6846-6855). Without being bound by theory, it was hypothesized that a combination approach of the EGFRvIII-DBTE in conjunction with HER2-targeting DBTE (HER2-DBTE) would limit GBM immune escape in vivo. An EGFRvIII+/HER2+ heterogeneous GBM model was developed to demonstrate that a co-administration of EGFRvIIIDBTE and HER2-DBTE dramatically enhanced tumor suppression in the heterogeneous GBM challenge as compared with single DBTEs. These findings suggest targeting multiple antigens, as was studied here, likely provide more potent tumor targeting and limit immune escape in GBM as well as other diverse cancers supporting improved patient benefit.

A new EGFRvIII-DBTE was developed for study in therapeutic models of GBM, exploring its anti-tumor cytotoxicity, specificity, T cell activation, in vivo pharmacokinetics, and impact in GBM challenge models. This treatment exhibited durable in vivo expression of EGFRvIII-DBTE over 15 weeks, with killing activity and continued ability to lower and clear tumor burden in all treated animals in an intracranial challenge of GBM after a single dose. A dual tumor targeting approach was studied, combining EGFRvIII-DBTE and HER2-DBTE treatments in a heterogeneous challenge model. In this combination approach, treatment with two DBTEs exhibited impressive control of heterogeneous GBM tumors and mitigated immune escape in 80% of the challenged mice.

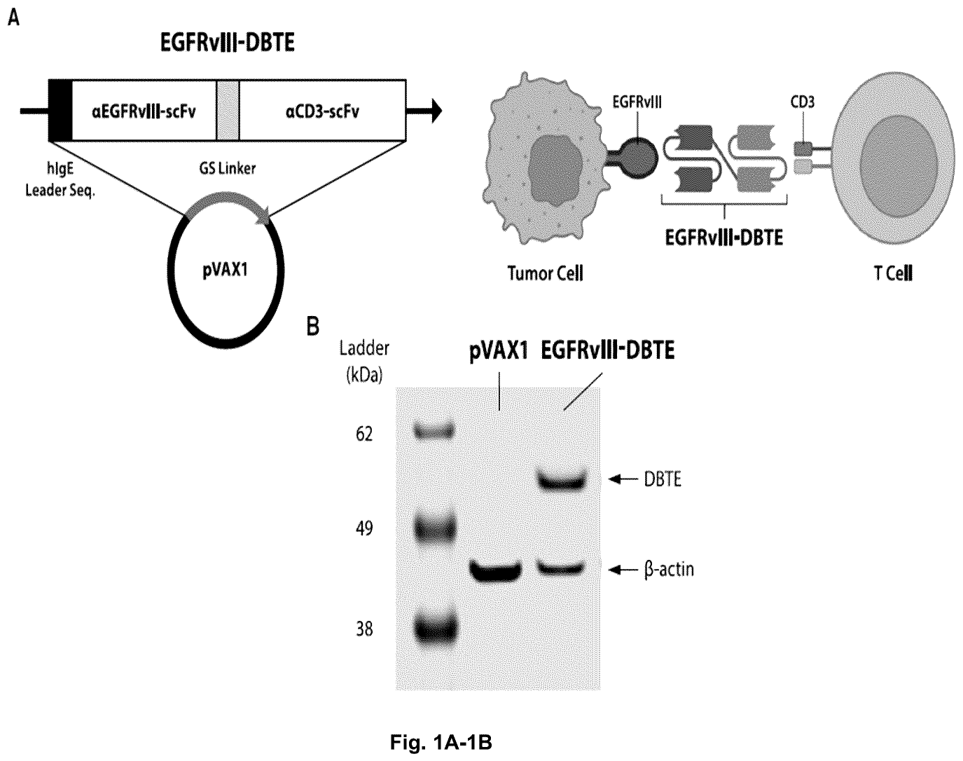

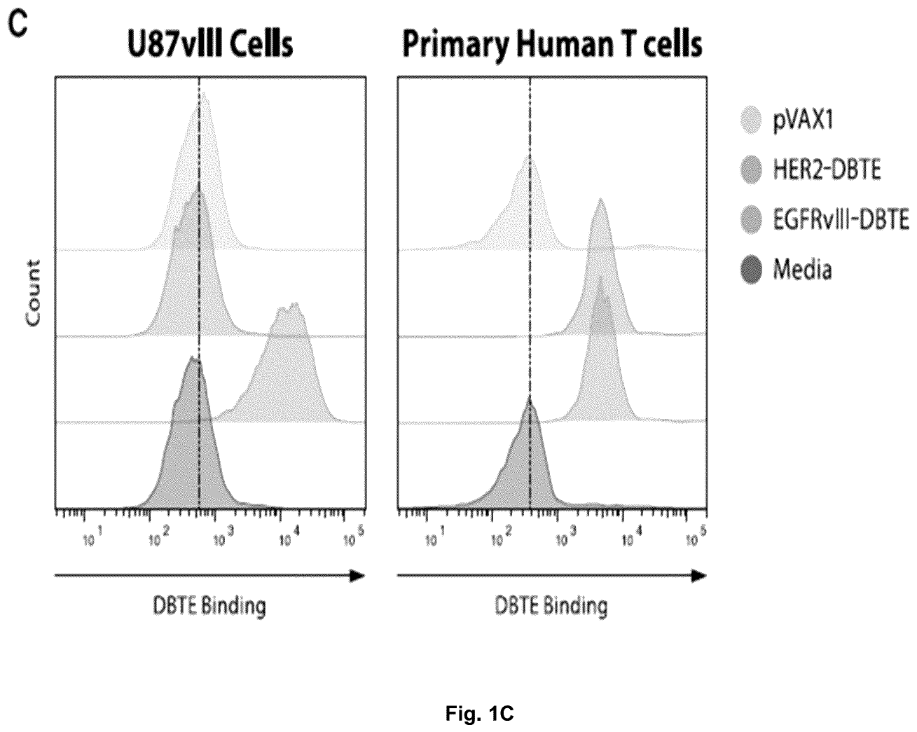

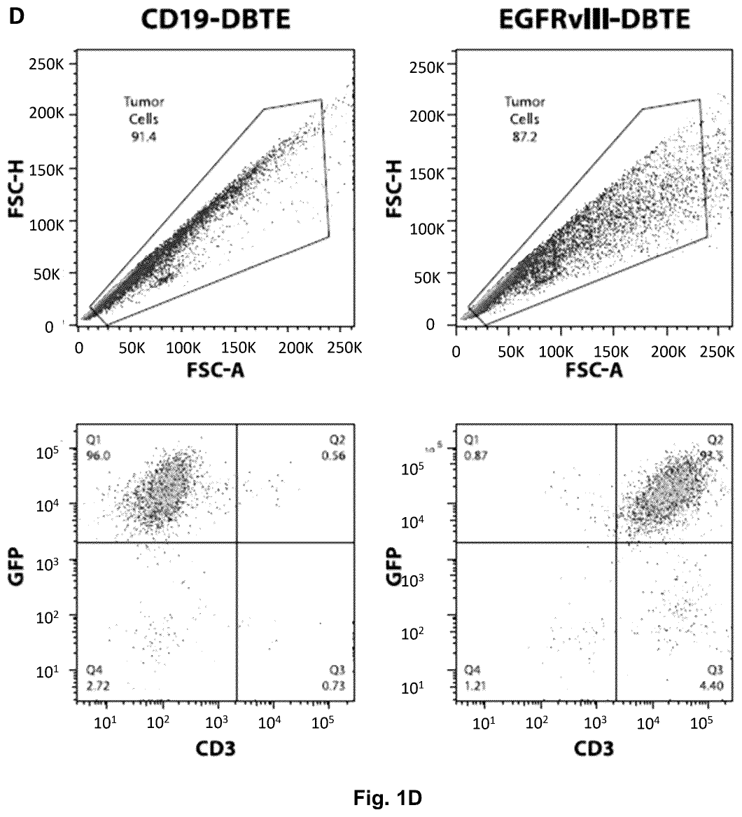

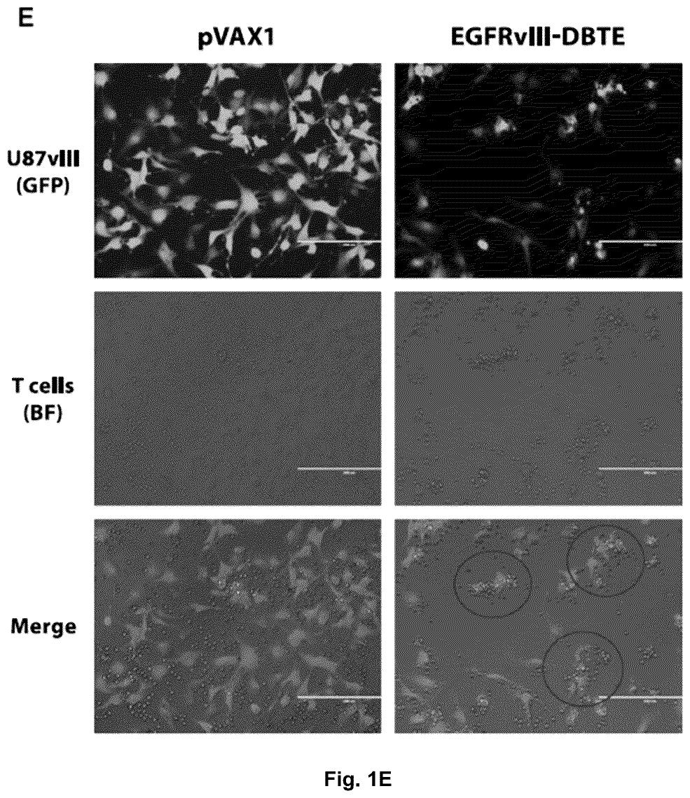

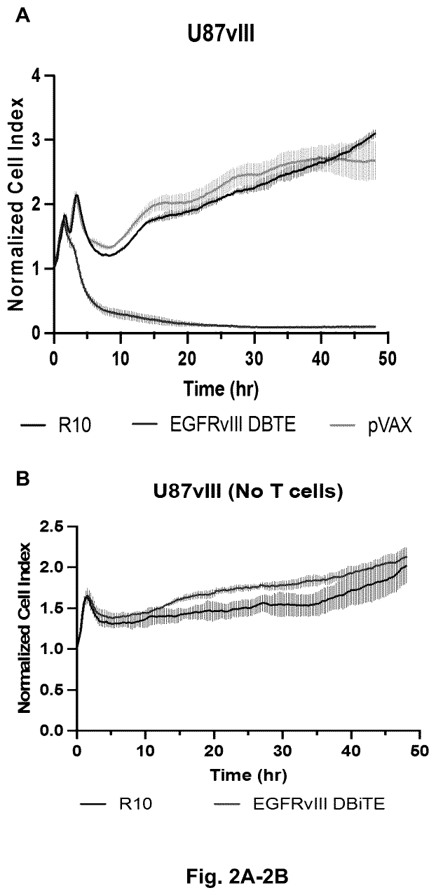

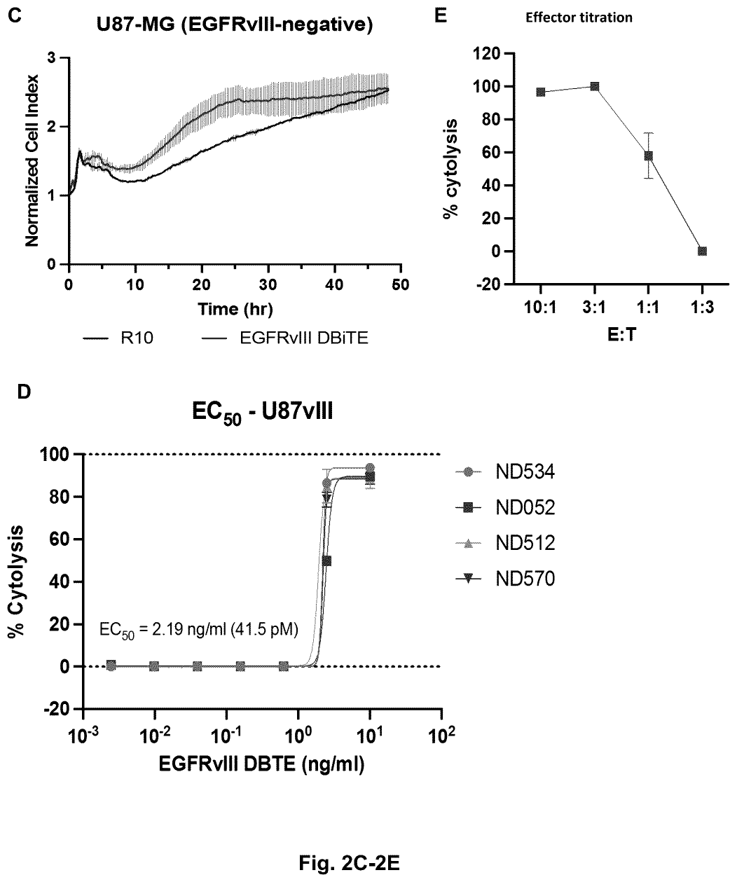

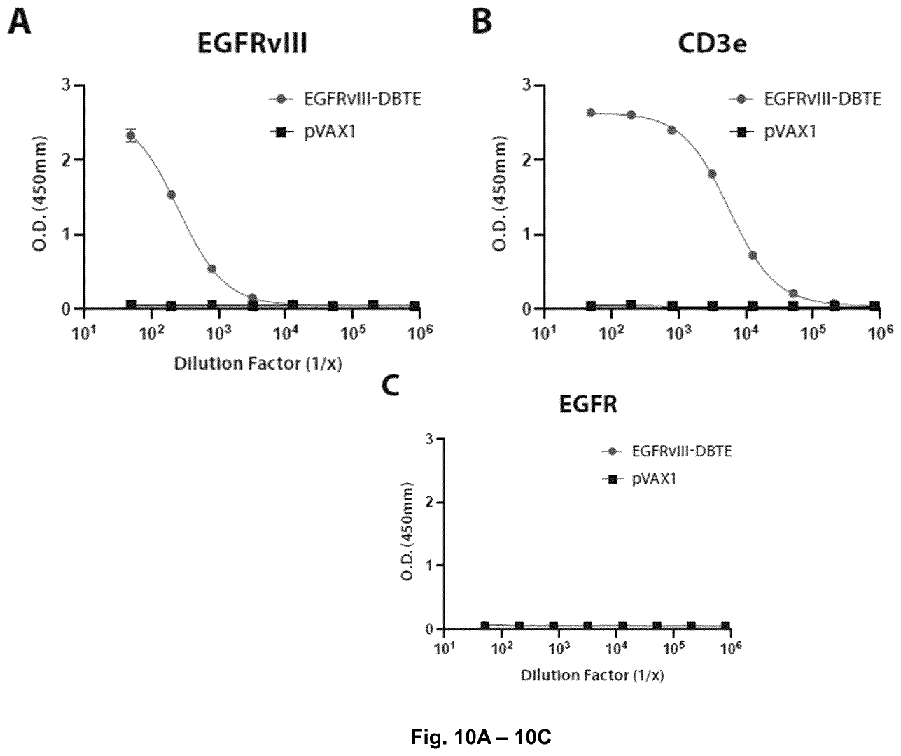

The detailed characterization of EGFRvIII in impact on tumor was also studied. The EGFRvIII-DBTE exhibited specific binding activities and cytotoxicity against EGFRvIII-expressing GBM cells (FIGS. 1C-1E and 2A), while not binding to wild-type EGFR (FIG. 10) or inducing anti-tumor cytotoxicity in the absence of either targets, EGFRvIII or CD3 (FIGS. 2B and 2C). Safety and toxicity are of concern in cancer immunotherapeutic agents in development. These data support the specificity of EGFRvIII-DBTE, reducing the potential of off-target toxicity. Also, the DNA vector itself has been reported to show low intrinsic immunogenicity and high safety profile in clinical trials (Trimble et al., 2015, Lancet 386, 2078-2088; Hollenberg et al., 2020, Hum. Vaccin. Immunother. 16, 1404-1412).

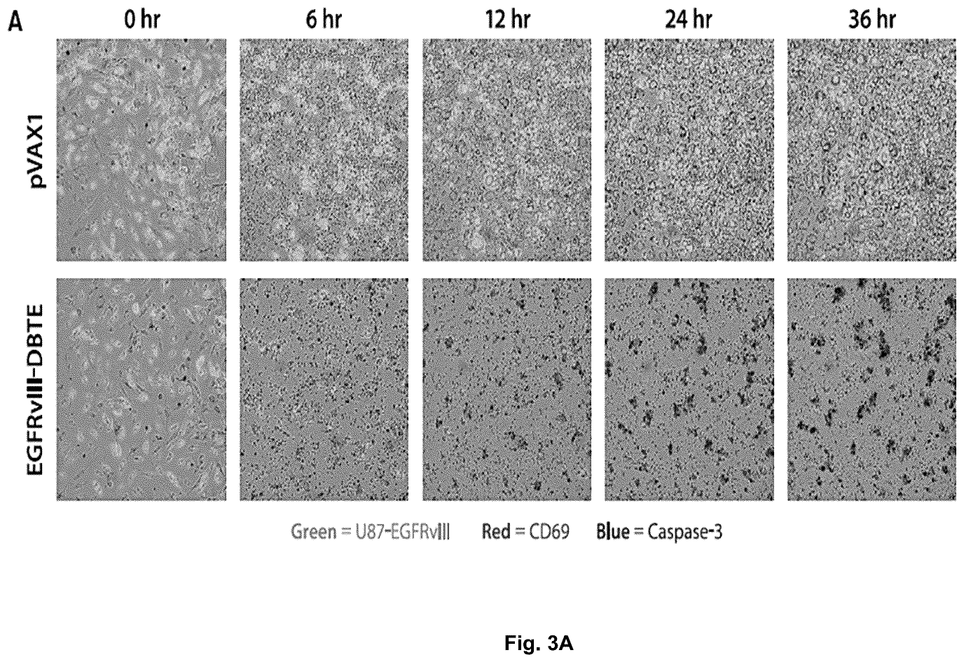

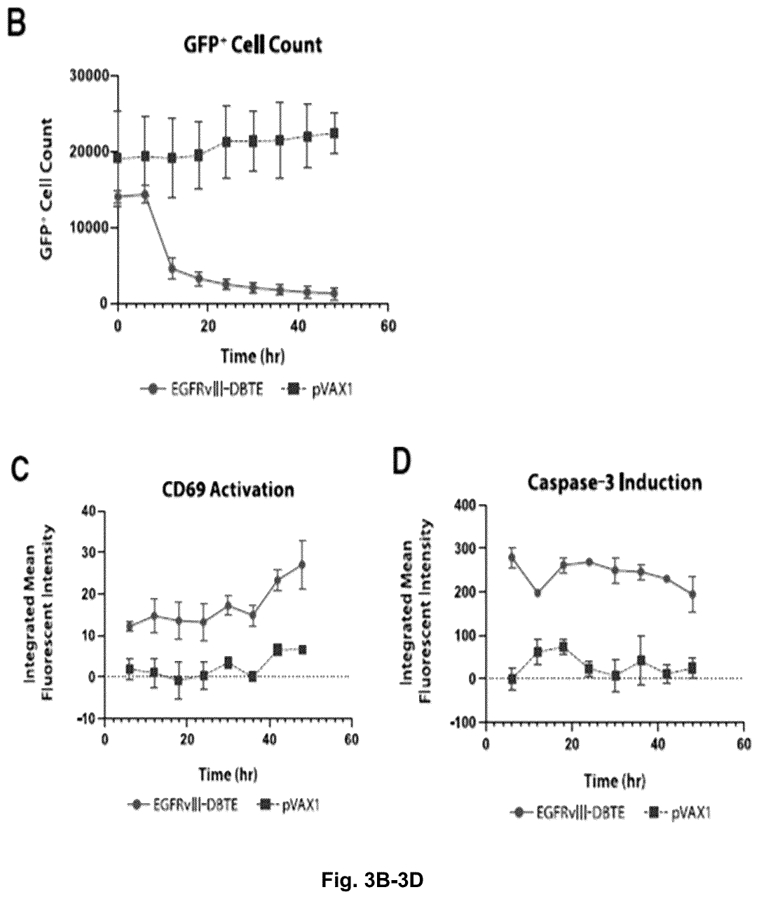

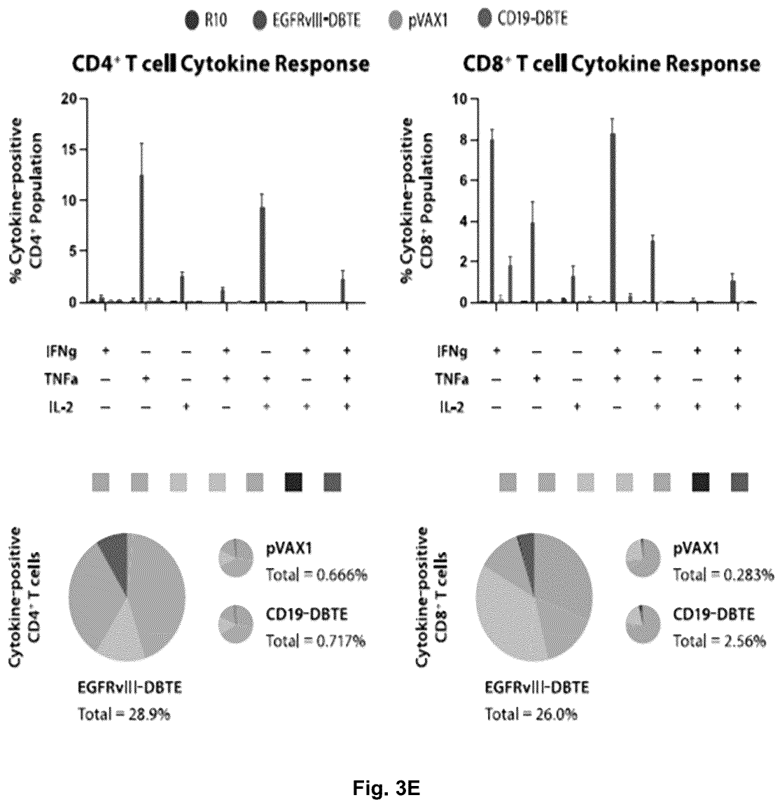

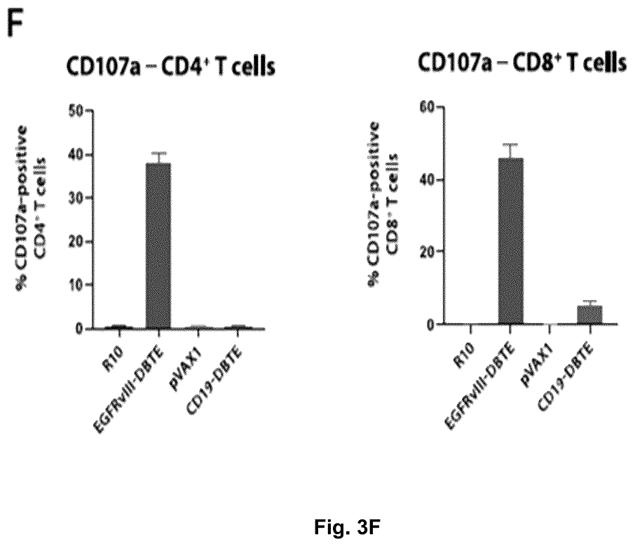

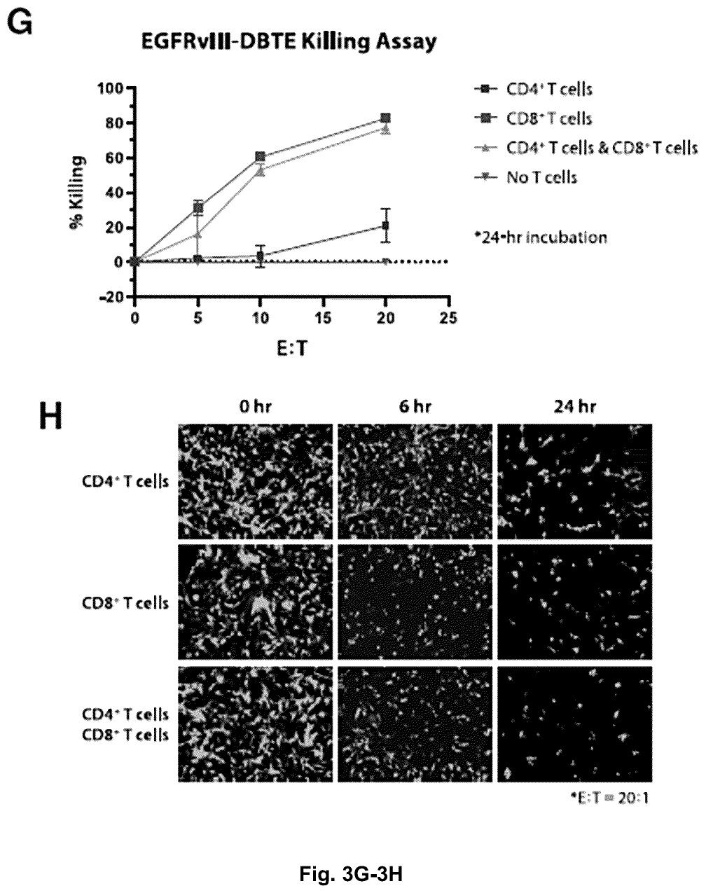

The T cell responses driven by the EGFRvIII-DBTE were characterized in an activation assay against U87vIII cells and observed that EGFRvIII-DBTE induced CD69 activation in T cells (FIG. 3A) and anti-tumor cytokine release in both CD8+ and CD4+ T cell populations (FIGS. 3E and 3F). One particularly important aspect was that CD4+ T cells also exhibited clear, but lower CD107a activation, supporting that cytolytic activities of CD4+ T cells are also induced by EGFRvIII-DBTE (FIG. 3F) (Betts et al., 2003, J. Immunol. Methods 281, 65-78). CD4+ T cells were analyzed in a tumor-killing assay and observed that EGFRvIII-DBTE induced antitumor cytotoxicity by isolated CD4+ T cells at an E:T ratio of 20:1 (FIGS. 3G and 3H). In the tumor microenvironment for immunecold tumors like GBM, CD8+ T cells often enter an inactivated state and instead CD4+ T cell tumor infiltration is observed. A high level of CD4+ tumor-infiltrating lymphocytes (TILs) with a low level of CD8+ TILs is associated with poor prognoses, which has been reported in glioma patients (Han et al., 2014, Br. J. Cancer 110, 2560-2568). The role of CD4+ T cells in cancer was thought to be primarily in priming immune response for CTLs. However, recent emerging evidence suggests that under some conditions, CD4+ T cells can participate in cytolytic activities against tumors (Tay et al., 2021, Cancer Gene Ther. 28, 5-17). These results expand on this work demonstrating that the stimulation using EGFRvIII-DBTE can induce CD4+ T cells to participate directly to kill target GBM and likely can be an important tool to recruit effector CD4+ T cells to target these tumors.

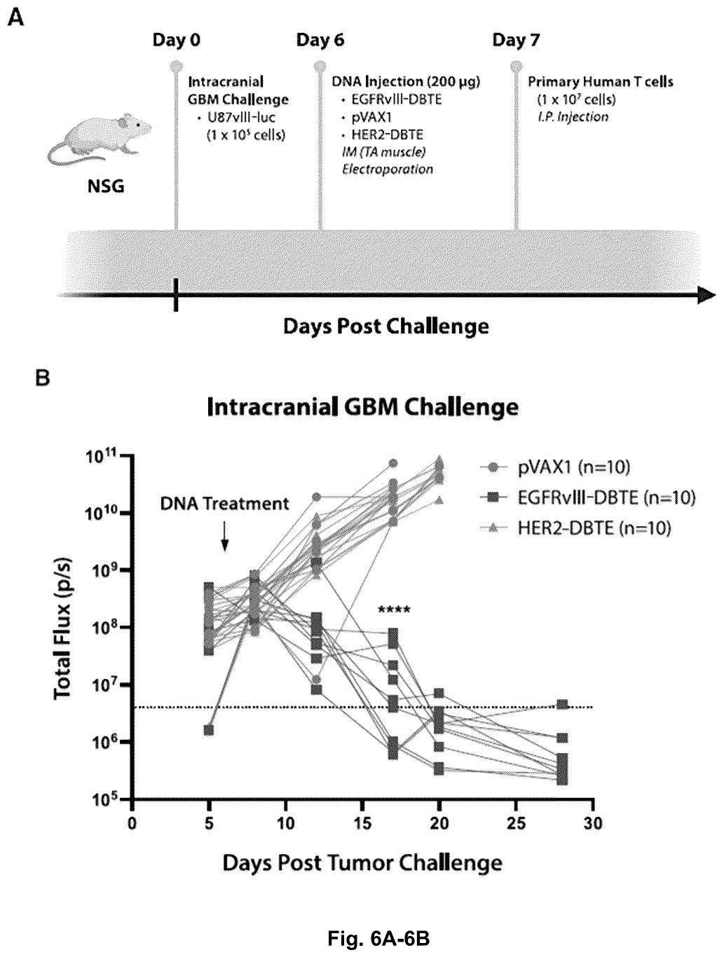

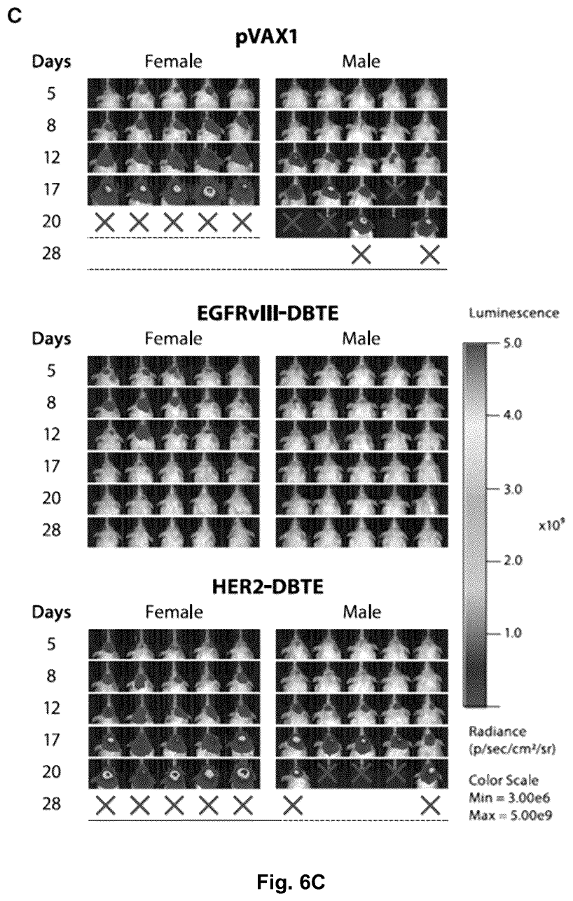

It has been suggested that the GBM microenvironment may impose challenges in in vivo delivery due to its immunosuppressive properties and the presence of the blood-brain barrier (BBB). However, recent reports suggest that BBB is impaired in GBM, which likely allows for increased passive diffusion, and the bioavailability of the peripherally delivered drugs in the brain remains of concern (Chen et al, 2018, Front. Immunol. 9, 1004; Butler et al., 2020, Adv. Exp. Med. Biol. 1296, 1-9). Thus, agents that offer efficacy at low concentrations are likely important for treatment of GBM. In a killing assay, it was observed that EGFRvIII-DBTE induced potent cytotoxicity against U87vIII cells with a dose with EC50 of 41.5 pM (FIG. 2D) and function at a low E:T ratio of 1:1 (FIG. 2E). This engineered EGFRvIII-DBTE is a potent T cell engager that maintains function in the modeled GBM microenvironment studied here. Potency in the intracranial challenge was demonstrated, observing that the EGFRvIII-DBTE coordinates T cells to the target and reduces tumor load in the challenge model, resulting in complete clearance of tumor in all treated animals by a single injection (FIGS. 6B-6D).

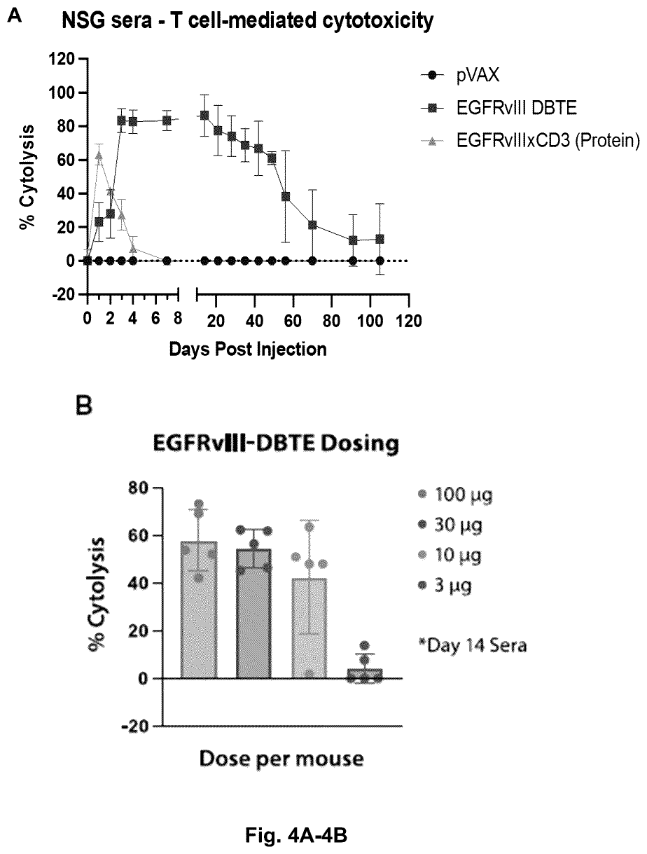

In an in vivo pharmacokinetics study, a single injection of EGFRvIIIDBTE exerted tumor-killing activity lasting more than 15 weeks (FIG. 4A). In contrast, a single injection of a recombinant EGFRvIIIxCD3 delivered peripherally exhibited cytotoxicity that lasted for just 4 days. Due to their short serum half-life, a proteinbased EGFRvIII-targeted BTE required multiple doses over 16 consecutive days to exert significant tumor regression and show 72% survival in an animal challenge in a recent study (Stemjak et al., 2021, Mol. Cancer Ther. 20, 925-933). However, in the intracranial challenge study described here, it was observed that a single injection of the EGFRvIII-DBTE exhibited complete tumor clearance with 100% (10 of 10 animals) survival (FIGS. 6B-6D), providing a simpler and potentially important additional treatment tool. It is possible that such genetic-based biologics could be dose and cost-sparing (Patel et al., 2020, BioDrugs. 34, 273-293). DBTEs may allow for tumor targeting and control for longer periods of time, potentially lowering treatment cost for patients.

The animal model used in which NSG mice are repopulated with primary human T cells is well established and a widely used model for development of bivalents and CAR-Ts, due to anti-drug antibody (ADA) responses in immunocompetent animal models (Dreier et al., 2003, J. Immunol. 170, 4397-4402; Friedrich et al., 2012, Mol. Cancer Ther. 11, 2664-2673; Pillarisetti et al., 2020, Blood Adv. 4, 4538-4549). ADA response is a significant challenge for antibody therapies in immune-competent models and does appear in the clinic at a detectable level. Humanized antibodies such as Blinatumomab, an FDAapproved BTE, have lower immunogenicity than mouse antibodies in human subjects, reporting<1% ADA responses in treated patients (Harding et al., 2012, MAbs 2, 256-265; Davda et al., 2019, J. Immunother. Cancer 7, 105). EGFRvIII-DBTE and HER2-DBTE described in this article are humanized antibodies, lowering the risk of ADA responses in humans. In vivo delivery by electroporation alone does not induce an ADA response, as we previously reported that a DNA/EP delivery in mice of a species-matched DNA-encoded monoclonal antibody resulted in a durable antibody expression over 10 weeks without detectable ADA responses (Patel et al., 2017, Nat. Commun. 8, 637).

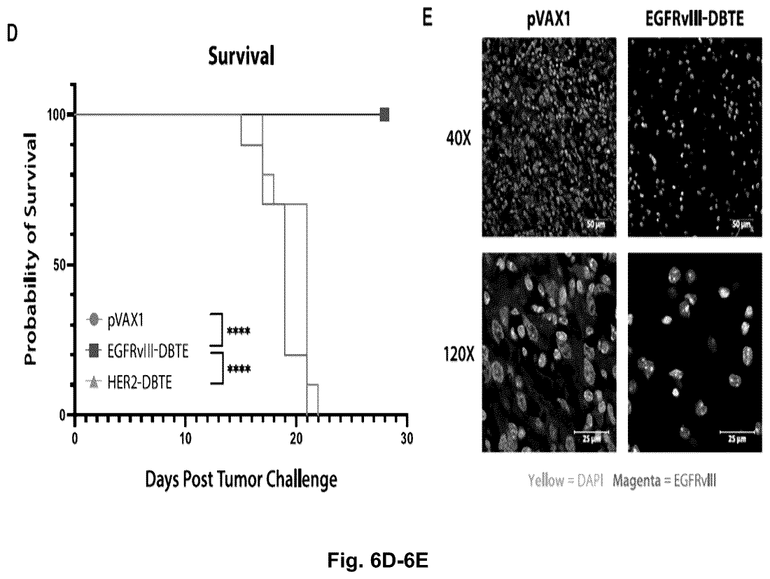

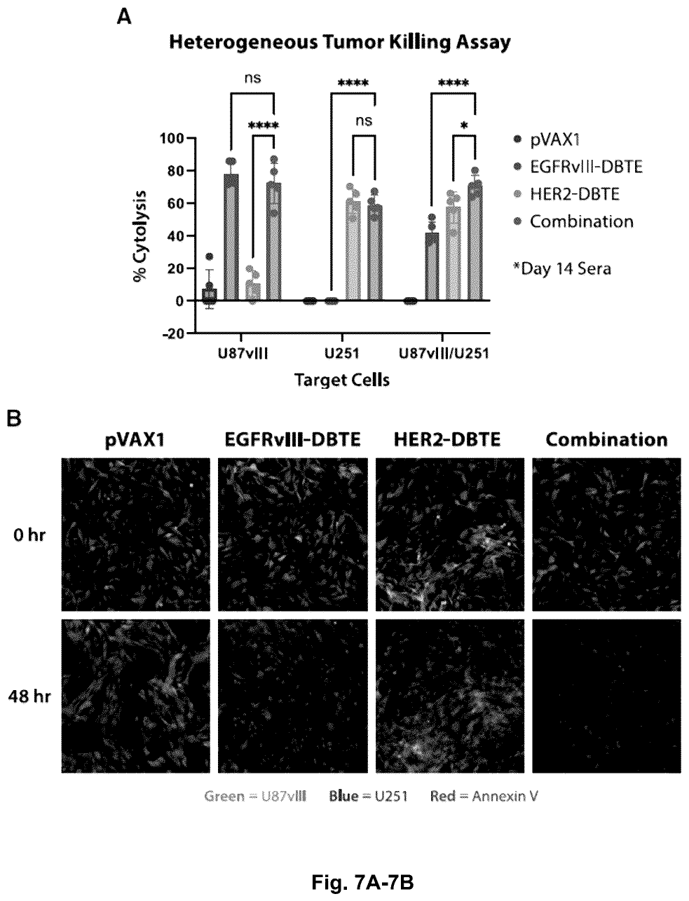

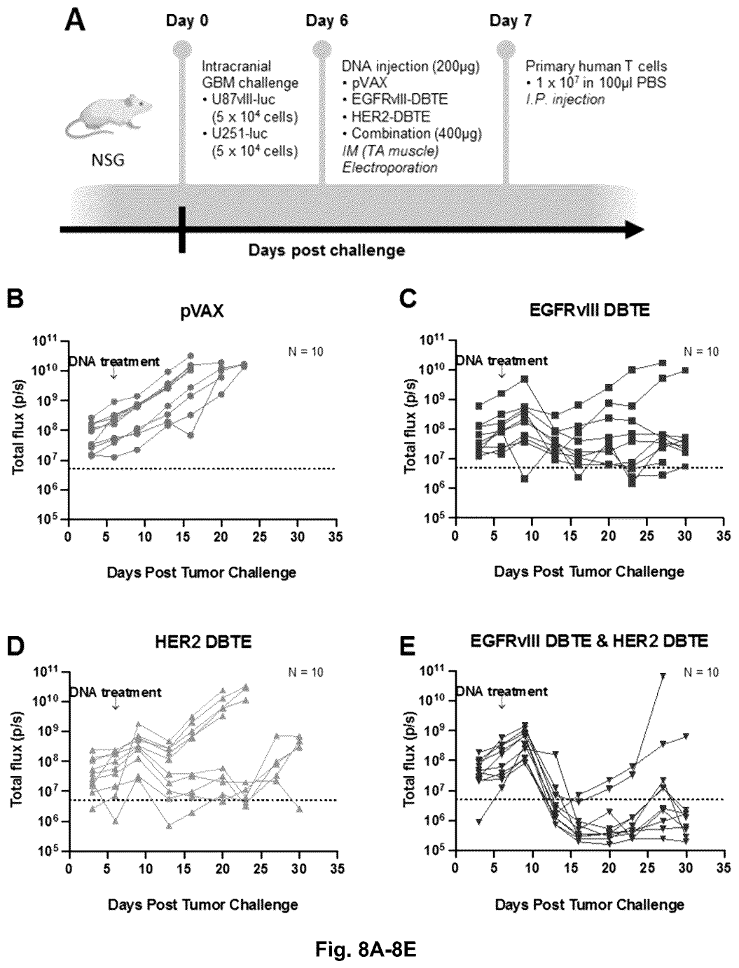

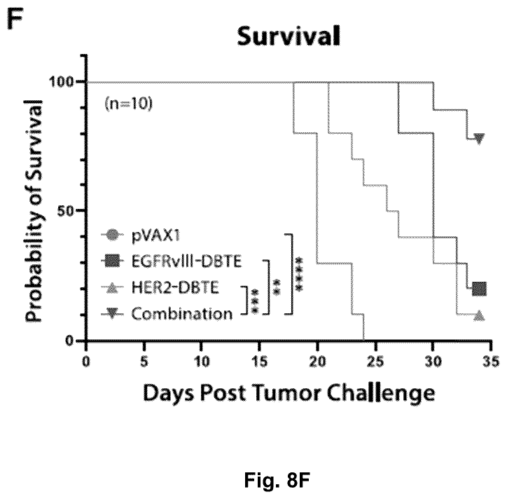

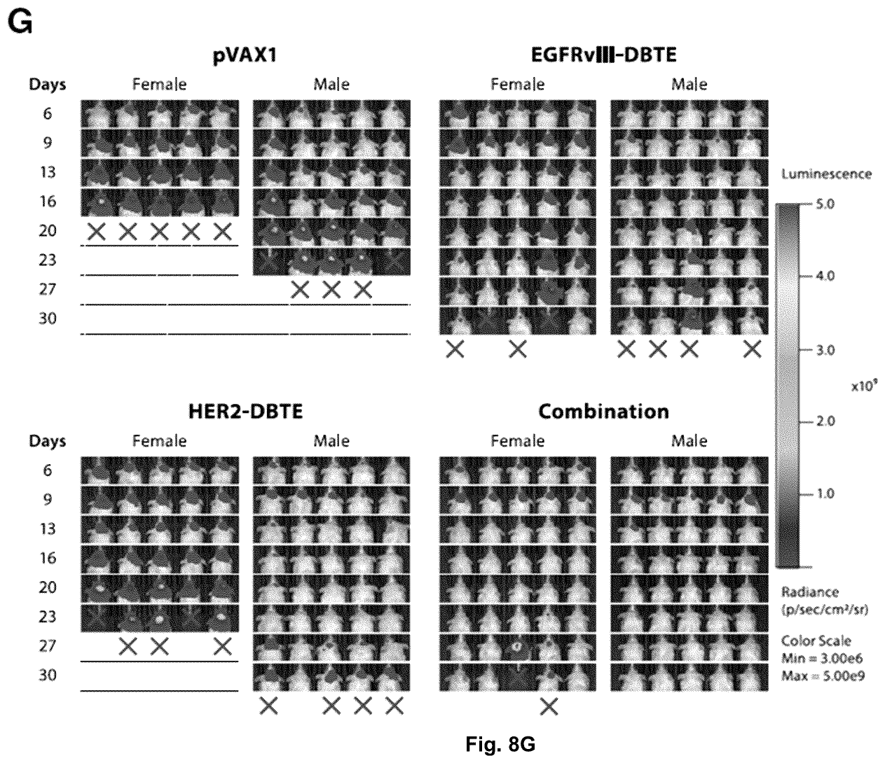

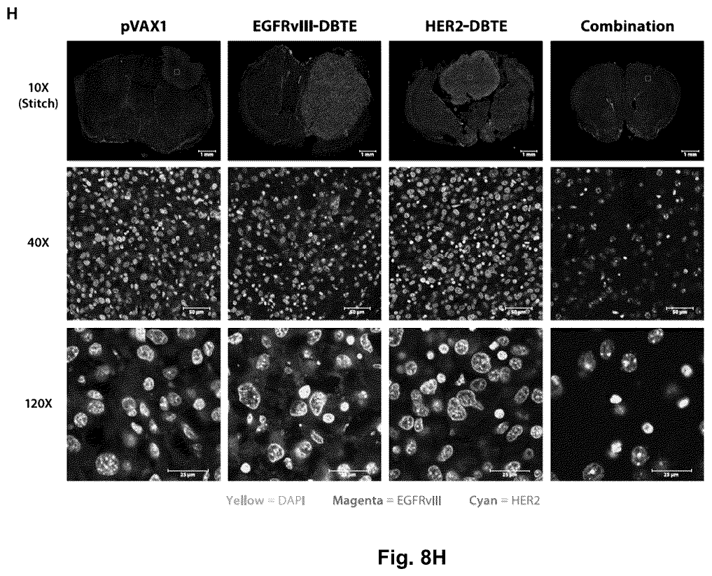

A challenge for immunotherapy for GBM cancer remains antigen heterogeneity. GBM displays various degrees of antigens such as EGFRvIII and HER2 in a heterogeneous manner (Saikali et al., 2007, J. Neurooncol. 81, 139-148; Liu et al., 2004, Cancer Res. 64, 4980-4986). Immunotherapies such as CAR-T and peptide vaccine, which targeted EGFRvIII alone, have not yet demonstrated significant clinical benefits in GBM patients thus far (O'Rourke et al., 2017, Sci. Transl. Med. 9, eaaa0984; Schuster et al., 2015, Neuro. Oncol. 17, 854-861). In single antigen-targeting therapies, tumor cells that are not recognized by the therapy likely use immune escape mechanisms by mutation of tumor antigen and selective survival of antigen-negative tumor subpopulations. Strategies that target multiple tumor antigens will likely help mitigate immune escape in this context. Tri-specific antibodies for dual tumor antigen targeting have been previously reported (Gantke et al., 2017, Protein Eng. Des. Sel. 30, 673-684; Tapia-Galisteo et al., 2022, Oncoimmunology 11, 2034355). However, these approaches used tumor models in which two antigens are homogeneously expressed and failed to address the impact of biologics in suppression of tumor escape. Here, co-delivery of multiple bispecific biologics in a heterogeneous in vivo tumor model is described wherein NSG mice are orthotopically challenged with a mixture of EGFRvIII-expressing tumor cells (U87vIII) and HER2-expressing tumor cells (U251). It is observed that sera from animals that received co-treatment of EGFRvIII-DBTE and a HER2-DBTE induced cytotoxicity against both EGFRvIII+ and HER2+ tumor populations (FIGS. 7A and 7B). In an intracranial heterogeneous GBM challenge, a single injection of the combined treatment of these DBTEs exerted enhanced tumor regression and improved survival as compared with single DBTE treatments or controls (FIGS. 8B-8F). Eighty percent of tumor bearing mice showed persistent tumor control and clearance if administered both DBTEs at the same time. This was not observed in mice treated with single DBTEs (FIG. 8F), supporting that the combined treatment with two DBTEs targeting two different antigens enhanced tumor suppression and improved morbidity in challenge. Examination of brain sections collected at the endpoints for each mouse revealed antigen escape in mice that received a single-agent therapy, but not in mice that received the combined treatment of both DBTEs (FIG. 8G). The two DBTEs effectively controlled tumor growth and mitigated antigen escape in the heterogeneous GBM challenge.

The simplicity in production and delivery of DBTEs suggests the importance of studying combination approaches targeting multiple tumor antigens. GBM expresses many other antigens that are receiving attention, such as IL13Ra2 and EphA2. A combination approach as is illustrated here could be expanded to target additional antigens, potentially further improving tumor control and advancing patient outcomes. Broadening treatment options for GBM and other cancer patients with combination therapies, potentially providing a personalized combination of DBTEs based on antigen expression profile of each patient, deserves additional study.

The materials and methods used for the experiments are now described

### Animals and Cell Lines

Male and female NSG mice were used in in vivo expression studies and GBM challenge models.

EGFRvIII-expressing U87-MG (U87vIII) cell line was generated by sequentially transducing U87-MG tumor cells (ATCC) with firefly luciferase lentivirus (PLV-10003, Cellomics Technology) and virus-containing media of Phoenix-AMPHO cells (CRL-3213, ATCC) transfected with pBMN-I-GFP embedding human EGFRvIII, which was generated by Genscript. Transduced cells were sorted by GFP expression and EGFRvIII expression was validated by anti-human EGFRvIII flow antibody (NBP2-50599, Novus Biologicals). U251-luc cell line was generated by transduction of U251-MG tumor cells (09,063,001, Millipore Sigma) with firefly luciferase lentiviral vector (PLV-10003, Cellomics Technology). DK-MG cells were obtained from Amsbio (CL 01008-CLTH). Tumor cells were kept in low passage number, cultured in MEM (or RPMI1640 for DK-MG) containing 10% heat-inactivated FBS and 100 U/mL penicillin/streptomycin, at 37° C. in a 5% CO₂ incubator.

Expi293F cell line (Thermo Fisher Scientific, A14527) was used for in vitro expression studies. Expi293F cells were cultured in Expi293 expression medium (A1435101, Thermo Fisher Scientific) and kept in suspension by an orbital shaker, at 37° C. in an 8% CO₂ incubator.

Primary human T cells were obtained from healthy donors at Human Immunology Core at University of Pennsylvania by negative selection using RosetteSep Human T cell isolation kit (Stemcell, #15061). T cells were cultured in RPM11640 containing 10% heat-inactivated FBS and 100 U/mL penicillin/streptomycin, at 37° C. in a 5% CO₂ incubator. In GBM challenge studies, T cells were activated and expanded with T cell activation/expansion kit (130-091-441, Miltenyi Biotec) and recombinant IL-2 (130-097-745, Miltenyi), following the manufacturer's protocol.

### Design of EGFRvIII-DBTE and HER2-DBTE

EGFRvIII-DBTE was designed by encoding a codon-optimized sequence of EGFRvIII-binding scFv (Johnson et al., 2015, glioblastoma. Sci. Transl Med. 7, 275ra222) fused with humanized CD3-binding scFv (clone UCHT-1) by a GS linker. Human IgE leader sequence was added to the N terminus of the construct. Altogether, the construct was subcloned into a modified pVAX1 expression vector. Previously described HER2-DBTE (Perales-Puchalt et al, 2019, JCI Insight 4, e126086) is composed of HER-binding scFv fused with CD3-binding scFv.

### In Vitro Expression of DBTEs

For in vitro expression of DBTEs, Expi293F cells were transfected with DBTE constructs by using ExpiFectamine 293 transfection kit (A14524 Thermo Fisher Scientific), following the manufacturer's protocol. Supernatants were collected at day 5 of transfection.

### Western Blot

The total protein concentration of the supernatant samples from DBTE-transfected Expi293F cells were quantified using a bicinchoninic acid assay (Pierce, Thermo Fisher Scientific). Ten micrograms of supernatant samples were loaded on a 4%-12% Bis-Tris SDSPAGE gel (NuPAGE, Thermo Fisher Scientific). The gel was transferred to a PVDF membrane using the iBlot 2 system (Thermo Fisher Scientific). The membrane was blocked in Intercept (PBS) blocking buffer (Licor) and then probed with a donkey anti-human IgG H+L secondary antibody (Licor) diluted 1:15,000 in Intercept T20 (PBS) antibody diluent (Licor). The membrane was scanned with Odyssey CLx imaging system (Licor). Western blotting was performed three times.

### Quantitative ELISA

Ninety-six-well plates (Fisher) were coated with anti-human F(ab′)2 antibody (Novus Bio) and incubated overnight at 4° C. The plates were blocked in PBS, 10% FBS, for 1 h and the diluted samples and standards were added for 1 h. Then they were probed with 1:5,000 anti-human F(ab′)2 antibody, horseradish peroxidase-conjugated (Jacksonimmuno Research) for 1 h. The plates were developed using TMB solution (ThermoFisher) for 10 min and stopped using 2N H₂SO₄ solution. The optical densities were measured at 450 nm using plate scanner (BioTek Synergy 2). The concentration of samples was determined based on the standard curve (4-parameter sigmoidal) using purified bispecific antibodies as standards. The purified bispecific antibodies were generated by Genscript by CHO transfection followed by purification using 6×HisTag at N terminus, which then was removed by protease. Quantitative ELISA experiments were performed with three replicates of each sample and standard.

### T Cell-Mediated Cytotoxicity Assay

U87vIII cells or U251-luc cells were plated on 96-well E-plate (ACEA biosciences) at 1×10⁴ cells/well in 100 mL RPMI 1640 medium containing 10% FBS (R10) and incubated at 37° C. overnight. Pre-treatment cell viability of the target cells was monitored by xCelligence RTCA eSight machine for 18 to 20 h. Primary human T cells were rested at 37° C. overnight in R10 and added to the target cells at various effector to target ratios together with DBTE-containing supernatant (10 ng/mL) or mouse serum (diluted 1:10) in a total volume of 100 mL. The cell viability was monitored with xCelligence RTCA eSight for 48 h. The cell viability of each assay well was normalized to the last cell index of pre-treatment incubation. Percent cytolysis was plotted as the percent difference of cell indices from the baseline (target cells with T cells only) at each time point. For fluorescent imaging, human CD69 antibody conjugated with Alexa Fluor 647 (FAB23591R, R&D Systems) and caspase-3 blue dye (SCT102, Millipore Sigma) were added at 10 mg/mL to the wells upon addition of effector cells. Bright field images and fluorescent (green, blue, red) images were taken with xCelligence RTCA eSight. T cell-mediated cytotoxicity assays were performed with three replicates for in vitro samples and five replicates for in vivo samples.

### Flow Cytometry

U87vIII cells were plated on 96-well plate (ThermoFisher) at 1×10⁴ cells/well in 100 μl RPMI 1640 medium containing 10% FBS (R10) and incubated at 37° C. overnight.

Primary human T cells were rested overnight in a 37° C. incubator and 5% CO₂ and added to the target cells together with DBTE-containing supernatant in a total volume of 100 μl. A 1× Protein transport inhibitor cocktail (eBioSciences, 00-4980-03) and CD107a antibody conjugated to PE-Cy7 (clone H4A3, Biolegend) were added to the wells. After 24-h incubation in a 37—C incubator and 5% CO₂, T cells were collected and washed with PBS. T cells were first incubated with Live/Dead viability stain (Zombie Yellow, Biolegend) diluted 1:1,000 in PBS for 10 min, and then CD4 conjugated to BV510 (clone OKT4, Biolegend) and CD8 conjugated to APC-Cy7 (clone SKI, Biolegend) diluted 1:100 in PBS with 1% FBS for 30 min. Cells were then fixed and permeabilized using Cytofix/Cytoperm reagents (554,714, BD Biosciences). Further intracellular staining was performed using IFN-γ conjugated to AF700 (clone B27, Biolegend), TNF-a conjugated to AF488 (clone MAb11, Biolegend), and IL-2 conjugated to PerCP/Cy5.5 (clone MQ1-17H12, Biolegend) diluted 1:100 in Perm/Wash buffer (554,723, BD Biosciences) for 1 h. Single stain and fluorescence minus one (FMO) controls were included for gating. Samples were analyzed using a BD LSR II flow cytometer and data were analyzed using FlowJo 10 software. Boolean gating was performed on T cell populations specifically secreting IFN-γ, TNF-α, and/or IL-2. Flow cytometry experiments were performed with three replicates of each sample.

### DBTE Treatment in Mice

For in vivo expression studies and tumor challenge studies, Mice received intramuscular injections (100 μg/site DNA plasmid) in tibialis anterior (TA) muscles of EGFRvIII-DBTE, HER2-DBTE, or pVAX1 DNA plasmid co-formulated with hyaluronidase (200 U/L, Sigma Aldrich, Saint Louis, MO), followed by electroporation (IM-EP) using the CELLECTRA 3P adaptive constant current device (Inovio Pharmaceuticals, Plymouth Meeting, PA). Serum was collected longitudinally to monitor in vivo expression.

### Mouse Xenograft Studies

In heterotopic GBM challenge studies, male and female NSG mice were inoculated with GBM tumors via subcutaneous injection of U87vIII (5×10⁵ cells in 100 μL of PBS) in the right flank. Tumor size was measured longitudinally with a digital caliper and the volume was calculated using the formula, V=(W²×L)/2. When the tumor size reached 50 mm³, the mice received DNA treatment and an i.p. injection of 1×10⁷ primary human T cells in 100 μL PBS. A second dose of DNA treatment was administered 7 days later. Tumors were scanned with IVIS Spectrum following i.p. injection of in vivo-grade luciferin (Promega). The mice were euthanized when tumor size reached 2000 mm³.

In the intracranial GBM challenge studies, male and female NSG mice received intracranial injection of 1×10⁵ tumor cells into the striatum. Mice were anesthetized with a cocktail of ketamine (Vedco, St. Joseph, MO, USA) and xylazine (Akom Animal Health, Lake Forest, IL, USA). Skull was trepanned with a drill 1 mm posterior to the bregma and 2 mm lateral to the midline. At 2.5 mm in depth, a 2-μL injection of 1×10⁵ tumor cells was inoculated over 2 min using a stereotactic frame and automatic syringe pump (Stoelting Co., Wood Dale, IL, USA). The syringe was withdrawn slowly (0.5 mm/min) and then the incision was sutured (Ethicon Inc., Somerville, NJ, USA). Mice received antibiotic ointment over the incision and a subcutaneous injection of buprenorphine analgesic. Then mice were monitored for adverse responses and weight loss. Mice that lost 20% of initial weight were euthanized in CO₂ chamber. Randomization was performed prior to DNA treatments.

### Fluorescent Immunohistochemical Images of Murine Brain Sections

At endpoint of the studies, mouse brains were harvested and fixed by sequentially incubating in 10% formalin (Millipore Sigma, USA), 15% and 30% sucrose solutions. The specimens were embedded in O.C.T. compound and frozen rapidly in dry ice. Ten-micron coronal sections of the brain specimens were performed by Histotechnology core at the Wistar Institute. The frozen section slides were blocked with 5% normal goat serum in PBS and stained with 10 μg/mL anti-EGFRvIII murine antibody conjugated to AF647 (clone DH8.3; Novus Biologicals, USA) and 10 μg/mL anti-HER2 murine antibodies conjugated to AF555 (clone EP1045Y; Abcam, USA) and DAPI. The fluorescent confocal images of the sections were captured using Leica TCS SP8 confocal microscope.

### Statistics

The data was graphed and statistical analyses performed using GraphPad Prism 9.0 software (La Jolla, CA). Statistical comparisons included a two-way analysis of variance analysis (ANOVA), with correction for multiple comparisonswhich compares groups within each time point (simple effects within rows). Survival data was represented by a Kaplan-Meier survival curve and significance was calculated using a log rank test between each group. In all experiments, samples with a p value<0.05 were considered statistically significant. The line graphs represent individual animals, where indicated. Scatter plots display individual animals, the mean value, and error bars represent the standard deviation.

The experimental results are now described

### Design and In Vitro Expression of EGFRvIII-Targeted DNAencoded Bispecific T Cell Engager

To develop an EGFRvIII-targeted DNA-encoded bispecific T cell engager (EGFRvIII-DBTE), we identified variable fragment sequences for an EGFRvIII-binding antibody (Johnson et al., 2015, Sci. Transl Med. 7, 275ra222) and a humanized CD3-binding antibody (clone UCHT-1). The sequences were modified to generate scFv sequences through codon optimization specific for in vivo expression and fusion with a GS linker (Figure TA). For improved expression in mammalian cells, we added a human immunoglobulin (Ig)E leader sequence to the N terminus, as we have previously described (Perales-Puchalt et al., 2019, JCI Insight 4, e126086), and encoded this construct in a modified pVAX1 expression vector. This EGFRvIII-DBTE was expressed in vitro using an Expi293 expression system. The supernatant from transfection studies was examined by western blotting to initially confirm expression, using pVAX1 empty vector as a negative control (FIG. 1B).

### Generation of EGFRvIII-Expressing Tumor Cells

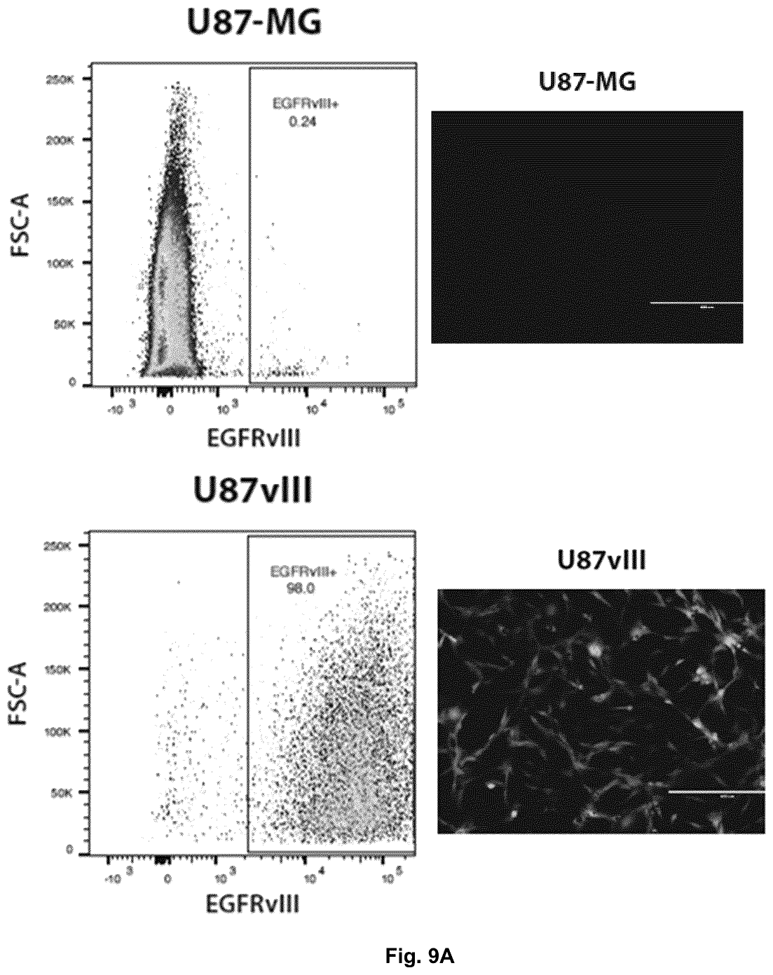

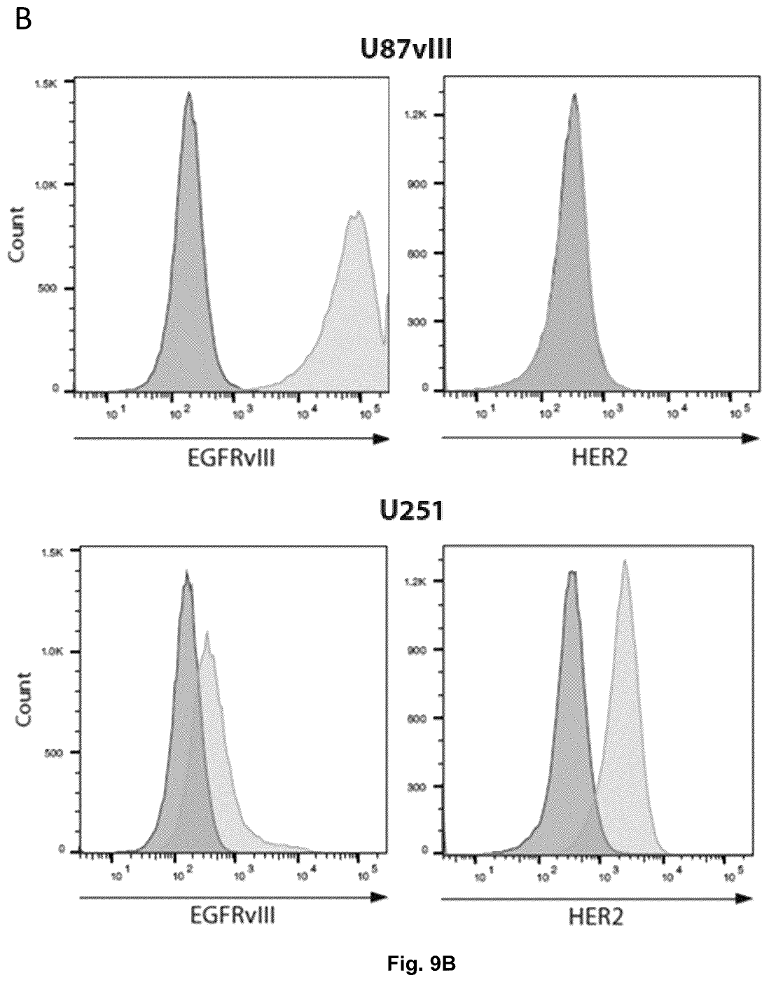

To develop a therapeutic model of GBM, we generated a GBM cell line stably expressing EGFRvIII. We transfected Phoenix-AMPHO cells with a DNA plasmid encoding extracellular sequence of EGFRvIII, which produced gamma-retrovirus containing the EGFRvIII construct and then used the virus-containing media to transduce U87-MG, an aggressive malignant glioma cell line to generate a GBM cell line stably expressing EGFRvIII. The construct included a GFP reporter, allowing for identification of tumor cells in vitro as well as in vivo. The EGFRvIII-expressing U87-MG cells (U87vIII) were sorted via GFP and subcloned allowing for generation of a homogeneous stable positive population that then was validated by flow cytometry (FIG. 9A).

### EGFRvIII-DBTE Binds EGFRvIII and CD3

To examine the binding properties of EGFRvIII-DBTE, U87vIII cells were incubated with EGFRvIII-DBTE and stained with an antihuman IgG F(ab′)2 fragment secondary antibody. For CD3-binding, primary human T cells were used. Supernatants collected from empty vector pVAX1 and HER2-targeted DBTE (HER2-DBTE) were used as controls. By flow cytometry analysis, it was observed that EGFRvIIIDBTE engaged with both U87vIII and human T cells (FIG. 1C). Binding specificity was also confirmed by ELISA in which EGFRvIII-DBTE did not bind to wild-type EGFR (FIG. 10). EGFRvIII-DBTE's ability to form an immunological synapse between the target and effector cells was examined by co-incubating U87vIII cells and T cells in the presence of EGFRvIII-DBTE and examined cultures for T cells engaging with U87vIII cells. A gate was established on the tumor population and a double-positive population of GFP (U87vIII) and CD3 (T cells) was observed in the presence of EGFRvIIIDBTE, indicating that T cells were engaging tumor cells (FIG. 1D). This engagement was not observed in the presence of an irrelevant control DBTE. In another assay, we plated U87vIII cells in a tissue culture plate with T cells and observed by fluorescent microscopy that EGFRvIII-DBTE induced T cells to cluster around the target cells (FIG. 1E). These data support that in vitro-expressed EGFRvIIIDBTE binds to both EGFRvIII and CD3, facilitating T cells to bind to target cells with high specificity.

### EGFRvIII-DBTE Cytotoxicity Against EGFRvIII-Expressing Tumor Cells

It was examined whether EGFRvIII-DBTE can induce T cell-mediated cytotoxicity against U87vIII cells. The xCelligence RTCA system was used, which uses gold biosensors at the bottom of the special plate to continuously and non-invasively measure the relative cell counts by impedance differential created by cell attachment to the plate. U87vIII cells were plated in a 96-well E-plate and primary human T cells were added at an E:T ratio of 10:1 and EGFRvIII-DBTE at 10 ng/mL. The viability of U87vIII cells was measured in real time for 48 h by xCelligence RTCA analyzer. As a result, EGFRvIII-DBTE induced potent T cell-mediated cytotoxicity against U87vIII cells (FIG. 2A). It was observed that EGFRvIII-DBTE did not induce cytotoxicity without T cells (FIG. 2B) or against EGFRvIII-negative U87 cells (FIG. 2C). To assess a half maximal effective concentration (EC50) value of EGFRvIII-DBTE, we examined % cytolysis using EGFRvIII-DBTE at a series of concentrations in 48-h tumor-killing assay. Using primary T cells from four different donors, we determined that EC50 of EGFRvIII-DBTE against U87vIII cells was potent at 2.19 ng/mL, which is equivalent to 41.5 pM (FIG. 2D). In addition, the potency of EGFRvIII-DBTE was examined in lower E:T ratios and observed that EGFRvIII-DBTE induced significant cytotoxicity at the low E:T ratio of 1:1 (FIG. 2E).

### Targeting Tumors by EGFRvIII-DBTE Drives T Cell Activation

To explore EGFRvIII-DBTE's ability to enhance T cell functions, activation markers and cytokine release were examined in primary T cells after stimulation by EGFRvIII-DBTE. In the tumor-killing assay described above, fluorochrome-conjugated CD69 antibody and caspase-3 dye was added and T cell activity was monitored. Upon addition of NOD scid gamma (NSG) mouse sera, which was treated with an intramuscular (IM) injection of 100 mg of EGFRvIII-DBTE followed by EP, CD69 activation was observed in T cells that was focused on target-bound T cells, as well as activation of the caspase-3 pathway in target cells (FIG. 3A). The cell counts of GFP+ target cells decreased significantly by 12 h when EGFRvIII-DBTE was added (FIG. 3B). CD69 activation (FIG. 3C) and caspase-3 induction (FIG. 3D) was rapid with initiation within 6 h and persistent throughout 48-h incubation, showing increasing signals for CD69. It was observed that T cells migrated toward the target tumor cells during CD69 activation.

Next, cytokine responses were examined in T cells using the tumor-killing assay. T cells were collected after a 24-h incubation with U87vIII cells in the presence of EGFRvIII-DBTE and then analyzed by flow cytometry. CD19-DBTE was used as an isotype control. It was observed that CD4+ and CD8+ T cell populations both exhibited increased secretion of interferon (IFN)-g, tumor necrosis factor (TNF)-a, and interleukin (IL)-2, which are associated with anti-tumor activities (FIG. 3E). CD8+ T cells displayed upregulation of IFN-g and TNF-α secretion while CD4+ T cells exhibited an upregulation of IL-2 and TNF-α secretion. Both CD4+ and CD8+ T cells showed activation of CD107a, a marker for degranulation, with a greater response observed in CD8+ T cells (FIG. 3F). CD4+ and CD8+ T cells were sorted and used as effector cells in a tumor-killing assay to examine their independent cytotoxicity. We observed robust cytotoxicity from CD8+ T cells (FIG. 3G). Importantly, CD4+ T cells also showed significant but lower cytotoxicity at a high E:T ratio of 20:1 (FIG. 3G). By fluorescent microscopy, we observed that the onset of tumor cytolysis was more rapid in CD8+ T cells than in CD4+ T cells (FIG. 3H). These data support that EGFRvIII-DBTE drives anti-tumor activation of both CD8+ and CD4+ T cells that can contribute to bispecific killing potential against tumor targets with slower kinetics.

### In Vivo Expression of EGFRvIII-DBTE

To determine functionality of EGFRvIII-DBTE expressed in vivo, we injected a single dose of EGFRvIII-DBTE or pVAX1 in the tibialis anterior (TA) muscle of NSG mice using electroporation (EP), as previously described.18 Sera were collected over a period of 105 days and EGFRvIII-DBTE activity was studied in 48-h T cellmediated cytotoxicity assays against U87vIII cells to monitor tumor killing over time. It was observed that a single injection of 100 mg of EGFRvIII-DBTE produced a durable expression in NSG mice of more than 100 days (FIG. 4A). For comparison, sera of NSG mice given an intraperitoneal (i.p.) injection of 100 mg of protein EGFRvIIIxCD3 BTE was included as a control. Cytotoxicity of the i.p.-delivered protein EGFRvIIIxCD3 BTE peaked in the first day but quickly declined and diminished after 4 days (FIG. 4A). In addition, NSG mice were treated with lower doses of EGFRvIIIDBTE and it was observed that the day 14 sera of the mice treated with as low as a 10-mg dose induced significant cytotoxicity against U87vIII cells (FIG. 4B). These results illustrate that a single injection of EGFRvIII-DBTE can produce durable and potent in vivo expression, which is not observed following a single dose of protein BTE infusion and is dose-sparing.

### Heterotopic Model of GBM

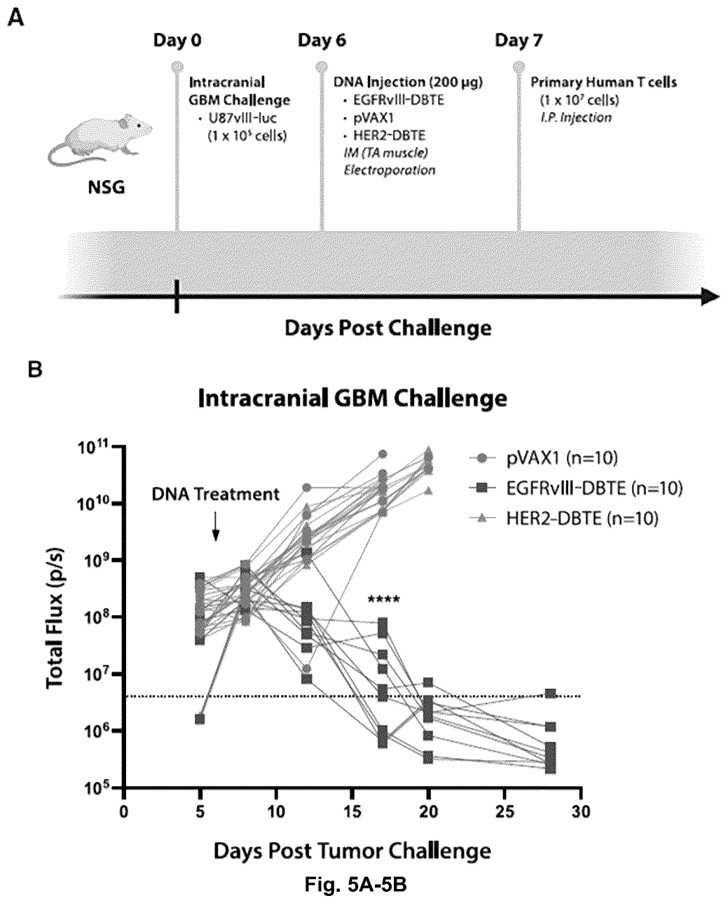

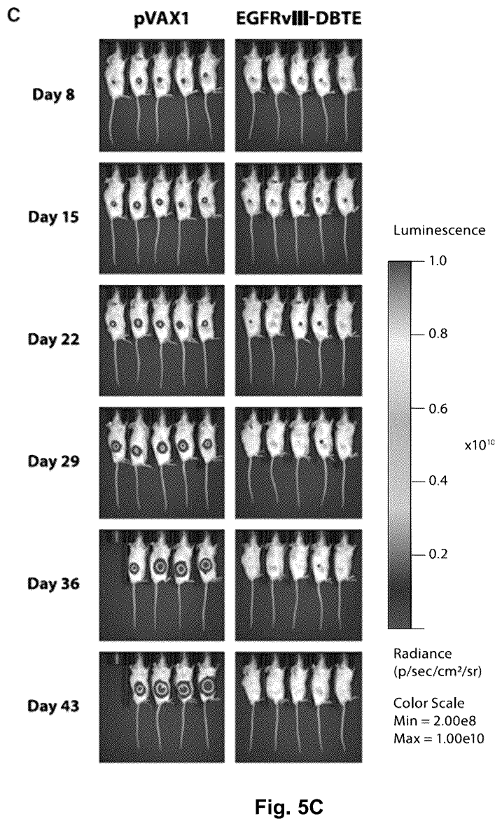

To evaluate in vivo cytotoxicity of EGFRvIII-DBTE, we conducted a GBM challenge in NSG mice by injecting U87vIII cells subcutaneously in the right flank. At day 8 when tumor size had grown to 50 mm3, the mice were given an IM injection of 100 mg EGFRvIIIDBTE or pVAX1 in the TA muscle followed by EP and an i.p. injection of primary human T cells. Seven days later, mice were treated with another injection of DNA/EP and continued to monitor the tumor sizes over time (FIG. 5A). It was observed that all five of five mice treated with EGFRvIII-DBTE demonstrated tumor regression, with four animals clearing the challenge, whereas zero of five mice treated with pVAX1 controlled their tumor growth (FIGS. 5B and 5C).

### EGFRvIII-DBTE Clears Tumor Burden in an Orthotopic Animal Model of GBM

To evaluate the efficacy of EGFRvIII-DBTE in an orthotopic model, an intracranial GBM challenge was conducted in NSG mice by injecting 1×10⁵ U87vIII-luc cells in the right hemisphere of NSG mice. At day 6, the mice were treated with EGFRvIII-DBTE, HER2-DBTE, or pVAX1 in the TA muscle followed by EP. At day 7, mice received an i.p. injection of primary human T cells (FIG. 6A). Tumor burden was monitored by IVIS Spectrum using in vivo-grade luciferin. It was observed that 10 of 10 mice treated with EGFRvIII-DBTE exhibited tumor clearance. None of 10 mice treated with pVAX1 or 10 mice treated with HER2-DBTE targeting irrelevant antigen in this model showed tumor regression (FIGS. 6B-6D). All 20 of the control animals succumbed to the challenge, demonstrating the specificity of the EGFRvIII-DBTE. This study in NSG mice did not continue beyond approximately 28 to 34 days due to onset of chronic graft versus host disease in the surviving animals, as is described for the used model (King et al., 2009, Clin. Exp. Immunol. 157, 104-118). Cryosections of the mouse brains were collected at the endpoints and examined by confocal microscopy for EGFRvIII expression in the tumor region of the brain. It was observed that the brains of EGFRvIII-DBTE-treated mice showed clearance of tumor burden as well as EGFRvIII expression, neither of which was observed in the brains of pVAX1-treated mice (FIG. 6E).

### EGFRvIII+/HER2+ Heterogeneous Model of GBM

A significant challenge for GBM immunotherapy remains the heterogeneity of antigen expression, which permits tumor escape in single-agent immunotherapeutic approaches.5,6,11 A strategy for overcoming this issue could be co-delivery of multiple DBTEs targeting additional antigens. Here we targeted both EGFRvIII and HER2, which are expressed in up to 30% and 80% of GBM cases, respectively.3,16 In this set of studies, we used EGFRvIII-DBTE in conjunction with previously described HER2-DBTE, which showed efficacy in a HER2-expressing tumor model.15 To develop an EGFRvIII+/HER2+ heterogeneous model of GBM, we chose U87vIII (EGFRvIII+/HER2+) and U251 cells (EGFRvIII+/HER2+) (FIG. 9B).

U87vIII and U251 cells were plated together in the same well in a 1:1 ratio mixture. Sera from mice treated with EGFRvIII-DBTE, HER2-DBTE, or combination of the two DBTEs was then added to the tumor cells along with primary T cells. After a 48-h incubation, the mice co-treated with both EGFRvIII-DBTE and HER2-DBTE exhibited enhanced cytotoxicity against heterogeneous tumor cells compared with sera of the mice treated with single DBTE (FIG. 7A). It was also observed that co-administration of the DBTEs did not impair killing of U87vIII cells when compared with EGFRvIII-DBTE or U251 cells when compared with HER2-DBTE, indicating co-delivery of the two DBTEs does not interfere with the expression and the tumor-killing capabilities of one another. In the heterogeneous tumor-killing assay, U251 cells were dyed with a cell-trace blue dye and observed by fluorescent microscopy that the mouse sera co-treated with two DBTEs induced apoptosis in both U87vIII (GFP) and U251 (blue) cell populations (FIG. 7B).

### Co-Delivery of EGFRvIII-DBTE and HER2-DBTE Enhanced Tumor Regression and Improved Survival in an Orthotopic Animal Model of Heterogeneous GBM

To further investigate the efficacy of a combined treatment of EGFRvIII-DBTE and HER2-DBTE, an intracranial challenge of heterogeneous GBM in NSG mice was developed and conducted. In this model, 5×10⁴ U87vIII-luc cells and 5×10⁴ U251-luc cells were implanted in a single injection in the right hemisphere of NSG mice, with five female and five male mice per group. On day 6 of challenge, animals were treated with a single 200-mg dose of pVAX1, EGFRvIII-DBTE, HER2-DBTE, or both DBTEs delivered in separate sites. All mice were given an i.p. injection of 1×10⁷ primary human T cells the following day (FIG. 8A). Tumor burden was monitored by IVIS Spectrum using in vivo-grade luciferin. Enhanced tumor regression was observed and survival in the group that received combined treatment of the two DBTEs over the groups that received single DBTE treatments (FIGS. 8B-8F). In the pVAX1-treated group, uncontrolled, aggressive tumor growths were observed in 10 of 10 mice (FIG. 8B). In the EGFRvIII-DBTE-treated group, seven of 10 mice showed moderate tumor control initially and two mice lost tumor control soon after treatment, while one mouse succumbed to challenge after initial tumor escape (FIG. 8C). In the HER2-DBTE-treated group, one mouse showed tumor regression while nine mice lost tumor control (FIG. 8D). In the combined treatment group, eight of 10 mice showed complete tumor regression while two mice exhibited tumor escape (FIG. 8E). At study completion on day 34, 80% of the mice that received both DBTEs survived the challenge, whereas 20% in the EGFRvIII-DBTE group, 10% in HER2-DBTE group, and 0% in pVAX1 group survived the heterogeneous GBM challenge (FIG. 8F). This study was limited to 34 days due to onset of chronic graft versus host disease in the surviving animals (King et al., 2009, Clin. Exp. Immunol. 157, 104-118).

TABLE 1
Synthetic Antibody Sequences

| SEQ | | |
|---|---|---|
| ID NO: | Sequence Type | Description |
| 1 | Nucleotide | EGFRvIII-DBTE |
| 2 | Amino Acid | EGFRvIII- DBTE |
| 3 | Nucleotide | EGFRvIII- DBTE with IgE leader |
| 4 | Amino Acid | EGFRvIII- DBTE with IgE leader |
| 5 | Nucleotide | Her2 DBTE |
| 6 | Amino Acid | Her2 DBTE DICE |
| 7 | Nucleotide | Her2 DBTE with IgE leader |
| 8 | Amino Acid | Her2 DBTE with IgE leader |

>EGFRVIII-DBTE_DNA_seq, no leader sequence
SEQ ID NO: 1
GATGTCGTGATGACTCAGAGCCCCGATTCCCTGGCCGTGTCCCTGGGCGAGAGAGCCACA
ATCAACTGCAAGAGCTCCCAGAGCCTGCTGGACTCTGATGGCAAGACCTACCTGAATTGG
CTGCAGCAGAAGCCAGGCCAGCCTCCAAAGCGGCTGATCAGCCTGGTGTCCAAGCTGGAT
AGCGGCGTGCCTGACAGATTCAGCGGCTCCGGCAGCGGCACCGACTTTACCCTGACAATC
AGCTCTCTGCAGGCCGAGGATGTGGCCGTGTATTACTGCTGGCAGGGCACCCACTTTCCT
GGCACCTTTGGCGGCGGCACCAAGGTGGAGATCAAGGGCGGCGGCGGCTCTGGCGGCGGC
GGCTCTGGCGGCGGCGGCAGCGGCGGCGGCGGCAGCGAGATCCAGCTGGTGCAGTCCGGC
GCCGAGGTGAAGAAGCCCGGCGAGTCCCTGAGAATCTCCTGCAAGGGCTCCGGCTTTAAT
ATCGAGGACTACTACATCCACTGGGTGCGGCAGATGCCAGGCAAGGGCCTGGAGTGGATG
GGCAGAATCGATCCAGAGAACGATGAGACCAAGTACGGCCCAATCTTCCAGGGCCACGTG
ACAATCAGCGCCGATACAAGCATCAATACCGTGTATCTGCAGTGGTCTTCCCTGAAGGCC
TCCGATACCGCCATGTATTACTGCGCCTTCAGAGGCGGCGTGTATTGGGGCCAGGGCACC
ACAGTGACAGTGTCCAGCGGCGGCGGCGGCTCTGAGGTGCAGCTGGTGGAGTCTGGCGGC
GGCCTGGTGCAGCCCGGCGGCAGCCTGAGACTGTCTTGCGCCGCCAGCGGCTATTCCTTC
ACAGGCTATACCATGAACTGGGTGCGGCAGGCCCCTGGCAAGGGCCTGGAGTGGGTGGCC
CTGATCAACCCATATAAGGGCGTGTCCACCTACAACCAGAAGTTCAAGGACCGCTTTACC
ATCTCTGTGGACAAGAGCAAGAATACAGCCTACCTGCAGATGAACAGCCTGAGAGCCGAG
GATACAGCCGTGTACTATTGCGCCAGATCCGGCTATTACGGCGACTCCGACTGGTACTTC
GACGTGTGGGGCCAGGGCACACTGGTGACCGTGTCTTCCGGCGGCGGCGGCAGCGGCGGC
GGCGGCTCCGGCGGCGGCGGCTCCGACATCCAGATGACCCAGTCCCCATCTTCCCTGTCC
GCCTCTGTGGGCGATAGAGTGACAATCACCTGCCGGGCCTCTCAGGACATCCGGAATTAT
CTGAACTGGTACCAGCAGAAGCCCGGCAAGGCCCCTAAGCTGCTGATCTACTATACCAGC
AGGCTGGAGAGCGGCGTGCCATCCAGATTCTCCGGCAGCGGCTCTGGCACAGACTATACC
CTGACCATCTCCAGCCTGCAGCCAGAGGATTTTGCCACCTATTACTGTCAGCAGGGGAAC
ACTCTGCCTTGGACATTCGGCCAGGGAACAAAAGTGGAAATCAAAAGTAGC

>EGFRVIII-DBTE_protein_seq, no leader
SEQ ID NO: 2
DVVMTQSPDSLAVSLGERATINCKSSQSLLDSDGKTYLNWLQQKPGQPPKRLISLVSKLD
SGVPDRFSGSGSGTDFTLTISSLQAEDVAVYYCWQGTHFPGTFGGGTKVEIKGGGGSGGG
GSGGGGSGGGGSEIQLVQSGAEVKKPGESLRISCKGSGFNIEDYYIHWVRQMPGKGLEWM
GRIDPENDETKYGPIFQGHVTISADTSINTVYLQWSSLKASDTAMYYCAFRGGVYWGQGT
TVTVSSGGGGSEVQLVESGGGLVQPGGSLRLSCAASGYSFTGYTMNWVRQAPGKGLEWVA
LINPYKGVSTYNQKFKDRFTISVDKSKNTAYLQMNSLRAEDTAVYYCARSGYYGDSDWYF
DVWGQGTLVTVSSGGGGSGGGGSGGGGSDIQMTQSPSSLSASVGDRVTITCRASQDIRNY
LNWYQQKPGKAPKLLIYYTSRLESGVPSRFSGSGSGTDYTLTISSLQPEDFATYYCQQGN
TLPWTFGQGTKVEIKSS

>EGFRVIII-DBTE _DNA_seq with IgE leader
SEQ ID NO: 3
ATGGACTGGACCTGGATTCTGTTCCTGGTGGCAGCCGCTACAAGAGTGCATTCCGATGTC
GTGATGACTCAGAGCCCCGATTCCCTGGCCGTGTCCCTGGGCGAGAGAGCCACAATCAAC
TGCAAGAGCTCCCAGAGCCTGCTGGACTCTGATGGCAAGACCTACCTGAATTGGCTGCAG
CAGAAGCCAGGCCAGCCTCCAAAGCGGCTGATCAGCCTGGTGTCCAAGCTGGATAGCGGC
GTGCCTGACAGATTCAGCGGCTCCGGCAGCGGCACCGACTTTACCCTGACAATCAGCTCT
CTGCAGGCCGAGGATGTGGCCGTGTATTACTGCTGGCAGGGCACCCACTTTCCTGGCACC
TTTGGCGGCGGCACCAAGGTGGAGATCAAGGGCGGCGGCGGCTCTGGCGGCGGCGGCTCT
GGCGGCGGCGGCAGCGGCGGCGGCGGCAGCGAGATCCAGCTGGTGCAGTCCGGCGCCGAG
GTGAAGAAGCCCGGCGAGTCCCTGAGAATCTCCTGCAAGGGCTCCGGCTTTAATATCGAG
GACTACTACATCCACTGGGTGCGGCAGATGCCAGGCAAGGGCCTGGAGTGGATGGGCAGA
ATCGATCCAGAGAACGATGAGACCAAGTACGGCCCAATCTTCCAGGGCCACGTGACAATC
AGCGCCGATACAAGCATCAATACCGTGTATCTGCAGTGGTCTTCCCTGAAGGCCTCCGAT
ACCGCCATGTATTACTGCGCCTTCAGAGGCGGCGTGTATTGGGGCCAGGGCACCACAGTG
ACAGTGTCCAGCGGCGGCGGCGGCTCTGAGGTGCAGCTGGTGGAGTCTGGCGGCGGCCTG
GTGCAGCCCGGCGGCAGCCTGAGACTGTCTTGCGCCGCCAGCGGCTATTCCTTCACAGGC
TATACCATGAACTGGGTGCGGCAGGCCCCTGGCAAGGGCCTGGAGTGGGTGGCCCTGATC
AACCCATATAAGGGCGTGTCCACCTACAACCAGAAGTTCAAGGACCGCTTTACCATCTCT
GTGGACAAGAGCAAGAATACAGCCTACCTGCAGATGAACAGCCTGAGAGCCGAGGATACA
GCCGTGTACTATTGCGCCAGATCCGGCTATTACGGCGACTCCGACTGGTACTTCGACGTG
TGGGGCCAGGGCACACTGGTGACCGTGTCTTCCGGCGGCGGCGGCAGCGGCGGCGGCGGC
TCCGGCGGCGGCGGCTCCGACATCCAGATGACCCAGTCCCCATCTTCCCTGTCCGCCTCT
GTGGGCGATAGAGTGACAATCACCTGCCGGGCCTCTCAGGACATCCGGAATTATCTGAAC
TGGTACCAGCAGAAGCCCGGCAAGGCCCCTAAGCTGCTGATCTACTATACCAGCAGGCTG
GAGAGCGGCGTGCCATCCAGATTCTCCGGCAGCGGCTCTGGCACAGACTATACCCTGACC
ATCTCCAGCCTGCAGCCAGAGGATTTTGCCACCTATTACTGTCAGCAGGGGAACACTCTG
CCTTGGACATTCGGCCAGGGAACAAAAGTGGAAATCAAAAGTAGC

>EGFRVIII-DBTE_protein_seq with IgE leader
SEQ ID NO: 4
MDWTWILFLVAAATRVHSDVVMTQSPDSLAVSLGERATINCKSSQSLLDSDGKTYLNWLQ
QKPGQPPKRLISLVSKLDSGVPDRFSGSGSGTDFTLTISSLQAEDVAVYYCWQGTHFPGT
FGGGTKVEIKGGGGSGGGGSGGGGSGGGGSEIQLVQSGAEVKKPGESLRISCKGSGFNIE
DYYIHWVRQMPGKGLEWMGRIDPENDETKYGPIFQGHVTISADTSINTVYLQWSSLKASD
TAMYYCAFRGGVYWGQGTTVTVSSGGGGSEVQLVESGGGLVQPGGSLRLSCAASGYSFTG
YTMNWVRQAPGKGLEWVALINPYKGVSTYNQKFKDRFTISVDKSKNTAYLQMNSLRAEDT
AVYYCARSGYYGDSDWYFDVWGQGTLVTVSSGGGGSGGGGSGGGGSDIQMTQSPSSLSAS
VGDRVTITCRASQDIRNYLNWYQQKPGKAPKLLIYYTSRLESGVPSRFSGSGSGTDYTLT
ISSLQPEDFATYYCQQGNTLPWTFGQGTKVEIKSS

-HER2-DBTE_DNA_seq, no leader sequence
SEQ ID NO: 5
ATGGACTGGACCTGGATACTGTTCCTGGTGGCCGCCGCCACACGGGTGCACTCCGACATC
CAGATGACCCAGTCCCCTAGCTCCCTGAGCGCCTCCGTGGGCGATAGAGTGACAATCACC
TGTAAGGCCAGCCAGGACGTGTCCATCGGCGTGGCCTGGTACCAGCAGAAGCCAGGCAAG
GCCCCCAAGCTGCTGATCTACAGCGCCTCCTATAGGTATACCGGCGTGCCATCCCGCTTT
TCTGGCTCTGGCTCCGGCACAGACTTCACCCTGACAATCTCCAGCCTGCAGCCAGAGGAC
TTCGCCACATACTATTGCCAGCAGTATTACATCTACCCCTACACCTTCGGCCAGGGCACC
AAGGTGGAGATCAAGAGAGGCGGCGGCGGCTCTGGCGGCGGCGGCAGCGGCGGCGGCGGC
TCCGAGGTGCAGCTGGTGGAGTCTGGCGGCGGCCTGGTGCAGCCAGGCGGCTCTCTGCGG
CTGAGCTGTGCCGCCTCCGGCTTCACCTTCACAGATTACACCATGGACTGGGTGCGCCAG
GCCCCCGGCAAGGGCCTGGAGTGGGTGGCCGATGTGAATCCCAATAGCGGCGGCTCTATC
TATAACCAGCGGTTTAAGGGCCGGTTTACCCTGTCTGTGGACCGCAGCAAGAATACCCTG
TACCTGCAGATGAACAGCCTGCGCGCCGAGGATACCGCCGTGTACTACTGCGCCAGAAAC
CTGGGCCCTAGCTTCTACTTTGATTACTGGGGCCAGGGCACCCTGGTGACAGTGTCCAGC
GGCGGCGGCGGCAGCGAGGTGCAGCTGGTGGAGTCCGGCGGCGGCCTGGTGCAGCCTGGC
GGCAGCCTGAGACTGTCCTGCGCCGCCTCCGGCTACTCTTTCACAGGCTATACCATGAAT
TGGGTGCGGCAGGCCCCAGGCAAGGGCCTGGAGTGGGTGGCCCTGATCAACCCCTATAAG
GGCGTGTCCACATACAACCAGAAGTTCAAGGACAGGTTTACCATCTCCGTGGATAAGTCC
AAGAATACCGCCTACCTGCAGATGAATTCCCTGCGGGCCGAGGATACAGCCGTGTACTAT
TGCGCCCGCTCCGGCTATTACGGCGATAGCGACTGGTATTTCGACGTGTGGGGCCAGGGC
ACACTGGTGACAGTGAGCTCCGGCGGCGGCGGCTCCGGCGGCGGCGGCTCTGGCGGCGGC
GGCTCCGATATCCAGATGACCCAGTCTCCCTCCAGCCTGTCCGCCTCTGTGGGCGACCGG
GTGACCATCACCTGTCGCGCCAGCCAGGATATCCGGAACTATCTGAACTGGTATCAGCAG
AAGCCCGGCAAGGCCCCAAAGCTGCTGATCTACTATACCTCTAGACTGGAGAGCGGCGTG
CCTTCCAGGTTTTCCGGCTCTGGCAGCGGCACCGACTATACCCTGACCATCTCCTCTCTG
CAGCCAGAGGATTTCGCCACATATTATTGTCAGCAGGGCAATACCCTGCCCTGGACATTT
GGCCAGGGCACAAAGGTGGAGATCAAG

-HER2-DBTE_protein_seq, no leader sequence
SEQ ID NO: 6
DIQMTQSPSSLSASVGDRVTITCKASQDVSIGVAWYQQKPGKAPKLLIYSASYRYTGVPS
RFSGSGSGTDFTLTISSLQPEDFATYYCQQYYIYPYTFGQGTKVEIKRGGGGSGGGGSGG
GGSEVQLVESGGGLVQPGGSLRLSCAASGFTFTDYTMDWVRQAPGKGLEWVADVNPNSGG
SIYNQRFKGRFTLSVDRSKNTLYLQMNSLRAEDTAVYYCARNLGPSFYFDYWGQGTLVTV
SSGGGGSEVQLVESGGGLVQPGGSLRLSCAASGYSFTGYTMNWVRQAPGKGLEWVALINP
YKGVSTYNQKFKDRFTISVDKSKNTAYLQMNSLRAEDTAVYYCARSGYYGDSDWYFDVWG
QGTLVTVSSGGGGSGGGGSGGGGSDIQMTQSPSSLSASVGDRVTITCRASQDIRNYLNWY
QQKPGKAPKLLIYYTSRLESGVPSRFSGSGSGTDYTLTISSLQPEDFATYYCQQGNTLPW
TFGQGTKVEIK

-HER2-DBTE_DNA_seq, with IgE leader
SEQ ID NO: 7
ATGGACTGGACCTGGATACTGTTCCTGGTGGCCGCCGCCACACGGGTGCACTCCGACATC
CAGATGACCCAGTCCCCTAGCTCCCTGAGCGCCTCCGTGGGCGATAGAGTGACAATCACC
TGTAAGGCCAGCCAGGACGTGTCCATCGGCGTGGCCTGGTACCAGCAGAAGCCAGGCAAG
GCCCCCAAGCTGCTGATCTACAGCGCCTCCTATAGGTATACCGGCGTGCCATCCCGCTTT
TCTGGCTCTGGCTCCGGCACAGACTTCACCCTGACAATCTCCAGCCTGCAGCCAGAGGAC
TTCGCCACATACTATTGCCAGCAGTATTACATCTACCCCTACACCTTCGGCCAGGGCACC
AAGGTGGAGATCAAGAGAGGCGGCGGCGGCTCTGGCGGCGGCGGCAGCGGCGGCGGCGGC
TCCGAGGTGCAGCTGGTGGAGTCTGGCGGCGGCCTGGTGCAGCCAGGCGGCTCTCTGCGG
CTGAGCTGTGCCGCCTCCGGCTTCACCTTCACAGATTACACCATGGACTGGGTGCGCCAG
GCCCCCGGCAAGGGCCTGGAGTGGGTGGCCGATGTGAATCCCAATAGCGGCGGCTCTATC
TATAACCAGCGGTTTAAGGGCCGGTTTACCCTGTCTGTGGACCGCAGCAAGAATACCCTG
TACCTGCAGATGAACAGCCTGCGCGCCGAGGATACCGCCGTGTACTACTGCGCCAGAAAC
CTGGGCCCTAGCTTCTACTTTGATTACTGGGGCCAGGGCACCCTGGTGACAGTGTCCAGC
GGCGGCGGCGGCAGCGAGGTGCAGCTGGTGGAGTCCGGCGGCGGCCTGGTGCAGCCTGGC
GGCAGCCTGAGACTGTCCTGCGCCGCCTCCGGCTACTCTTTCACAGGCTATACCATGAAT
TGGGTGCGGCAGGCCCCAGGCAAGGGCCTGGAGTGGGTGGCCCTGATCAACCCCTATAAG
GGCGTGTCCACATACAACCAGAAGTTCAAGGACAGGTTTACCATCTCCGTGGATAAGTCC
AAGAATACCGCCTACCTGCAGATGAATTCCCTGCGGGCCGAGGATACAGCCGTGTACTAT
TGCGCCCGCTCCGGCTATTACGGCGATAGCGACTGGTATTTCGACGTGTGGGGCCAGGGC
ACACTGGTGACAGTGAGCTCCGGCGGCGGCGGCTCCGGCGGCGGCGGCTCTGGCGGCGGC
GGCTCCGATATCCAGATGACCCAGTCTCCCTCCAGCCTGTCCGCCTCTGTGGGCGACCGG
GTGACCATCACCTGTCGCGCCAGCCAGGATATCCGGAACTATCTGAACTGGTATCAGCAG
AAGCCCGGCAAGGCCCCAAAGCTGCTGATCTACTATACCTCTAGACTGGAGAGCGGCGTG
CCTTCCAGGTTTTCCGGCTCTGGCAGCGGCACCGACTATACCCTGACCATCTCCTCTCTG
CAGCCAGAGGATTTCGCCACATATTATTGTCAGCAGGGCAATACCCTGCCCTGGACATTT
GGCCAGGGCACAAAGGTGGAGATCAAG

-HER2-DBTE_protein_seq, with IgE leader
SEQ ID NO: 8
MDWTWILFLVAAATRVHSDIQMTQSPSSLSASVGDRVTITCKASQDVSIGVAWYQQKPGK
APKLLIYSASYRYTGVPSRFSGSGSGTDFTLTISSLQPEDFATYYCQQYYTYPYTFGQGT
KVEIKRGGGGSGGGGSGGGGSEVQLVESGGGLVQPGGSLRLSCAASGFTFTDYTMDWVRQ
APGKGLEWVADVNPNSGGSIYNQRFKGRFTLSVDRSKNTLYLQMNSLRAEDTAVYYCARN
LGPSFYFDYWGQGTLVTVSSGGGGSEVQLVESGGGLVQPGGSLRLSCAASGYSFTGYTMN
WVRQAPGKGLEWVALINPYKGVSTYNQKFKDRFTISVDKSKNTAYLQMNSLRAEDTAVYY
CARSGYYGDSDWYFDVWGQGTLVTVSSGGGGSGGGGSGGGGSDIQMTQSPSSLSASVGDR
VTITCRASQDIRNYLNWYQQKPGKAPKLLIYYTSRLESGVPSRFSGSGSGTDYTLTISSL
QPEDFATYYCQQGNTLPWTFGQGTKVEIK

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nucleic acid molecule encoding one or more synthetic DNA encoded bispecific immune cell engager, wherein the more synthetic DNA encoded bispecific immune cell engager comprises at least one least one antigen binding domain, and at least one immune cell engaging domain.

2. The nucleic acid molecule of claim 1, wherein the antigen binding domain targets at least one antigen selected from the group consisting of epidermal growth factor receptor variant III (EGFRvIII), and human epidermal growth factor receptor 2 (Her2).

3. The nucleic acid molecule of claim 1, wherein the immune cell engaging domain targets a cell selected from the group consisting of a T cell, an antigen presenting cell, a natural killer (NK) cell, a neutrophil and a macrophage.

4. The nucleic acid molecule of claim 1, wherein the immune cell engaging domain targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95.

5. The nucleic acid molecule of claim 4, wherein the immune cell engaging domain targets CD3.

6. The nucleic acid molecule of claim 1 comprising a nucleotide sequence encoding one or more sequences selected from the group consisting of:
a) an amino acid sequence having at least about 90% identity over an entire length of the amino acid sequence to an amino acid sequence selected from the group consisting of SEQ ID NO:2, SEQ ID NO:4, SEQ ID NO:6, and SEQ ID NO:8;
b) a fragment of an amino acid sequence having at least about 90% identity over at least 65% of the amino acid sequence to an amino acid sequence selected from the group consisting of SEQ ID NO:2, SEQ ID NO:4, SEQ ID NO:6, and SEQ ID NO:8;
c) an amino acid sequence selected from the group consisting of SEQ ID NO:2, SEQ ID NO:4, SEQ ID NO:6, and SEQ ID NO:8; and
d) a fragment of an amino acid sequence comprising at least 65% of an amino acid sequence selected from the group consisting of SEQ ID NO:2, SEQ ID NO:4, SEQ ID NO:6, and SEQ ID NO:8.

7. The nucleic acid molecule of claim 1, selected from the group consisting of:
a) a nucleotide sequence having at least about 90% identity over an entire length of the nucleic acid sequence to a nucleotide sequence selected from the group consisting of SEQ ID NO:1, SEQ ID NO:3, SEQ ID NO:5, and SEQ ID NO:7;
b) a fragment of a nucleotide sequence having at least about 90% identity over at least 65% of the nucleic acid sequence to a nucleotide sequence selected from the group consisting of SEQ ID NO:1, SEQ ID NO:3, SEQ ID NO:5, and SEQ ID NO:7;
c) a nucleotide sequence selected from the group consisting of SEQ ID NO:1, SEQ ID NO:3, SEQ ID NO:5, and SEQ ID NO:7; and
d) a fragment of a nucleotide sequence comprising at least 65% of a nucleotide sequence selected from the group consisting of SEQ ID NO:1, SEQ ID NO:3, SEQ ID NO:5, and SEQ ID NO:7.

8. The nucleic acid molecule of claim 1, wherein the nucleotide sequence is operably linked to a nucleic acid sequence encoding an IgE leader sequence.

9. The nucleic acid molecule of claim 1, wherein the nucleic acid molecule comprises an expression vector.

10. A composition comprising at least one nucleic acid molecule of claim 1.

11. The composition of claim 10, further comprising a pharmaceutically acceptable excipient.

12. The composition of claim 10 comprising a first synthetic DNA encoded bispecific immune cell engager comprising an antigen binding domain targeting EGFRvIII and a second synthetic DNA encoded bispecific immune cell engager comprising an antigen binding domain targeting HER2.

13. The composition of claim 12, wherein the immune cell engaging domain for each of the first and second synthetic DNA encoded bispecific immune cell engager targets a cell selected from the group consisting of a T cell, an antigen presenting cell, a natural killer (NK) cell, a neutrophil and a macrophage.

14. The composition of claim 12, wherein the immune cell engaging domain for each of the first and second synthetic DNA encoded bispecific immune cell engager targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95.

15. The composition of claim 12, wherein the immune cell engaging domain for each of the first and second synthetic DNA encoded bispecific immune cell engager targets CD3.

16. The composition of claim 12, wherein the first synthetic DNA encoded bispecific immune cell engager comprising an antigen binding domain targeting EGFRvIII comprises a nucleotide sequence encoding one or more sequences selected from the group consisting of:
a) an amino acid sequence having at least about 90% identity over an entire length of the amino acid sequence to an amino acid sequence selected from the group consisting of SEQ ID NO:2 and SEQ ID NO:4;
b) a fragment of an amino acid sequence having at least about 90% identity over at least 65% of the amino acid sequence to an amino acid sequence selected from the group consisting of SEQ ID NO:2 and SEQ ID NO:4;
c) an amino acid sequence selected from the group consisting of SEQ ID NO:2 and SEQ ID NO:4; and
d) a fragment of an amino acid sequence comprising at least 65% of an amino acid sequence selected from the group consisting of SEQ ID NO:2 and SEQ ID NO:4; and
wherein the second synthetic DNA encoded bispecific immune cell engager comprising an antigen binding domain targeting HER2 comprises a nucleotide sequence encoding one or more sequences selected from the group consisting of:
e) an amino acid sequence having at least about 90% identity over an entire length of the amino acid sequence to an amino acid sequence selected from the group consisting of SEQ ID NO:6 and SEQ ID NO:8;
f) a fragment of an amino acid sequence having at least about 90% identity over at least 65% of the amino acid sequence to an amino acid sequence selected from the group consisting of SEQ ID NO:6 and SEQ ID NO:8;
g) an amino acid sequence selected from the group consisting of SEQ ID NO:6 and SEQ ID NO:8; and
h) a fragment of an amino acid sequence comprising at least 65% of an amino acid sequence selected from the group consisting of SEQ ID NO:6 and SEQ ID NO:8.

17. The composition of claim 12, wherein the first synthetic DNA encoded bispecific immune cell engager comprising an antigen binding domain targeting EGFRvIII comprises a nucleotide sequence selected from the group consisting of:
a) a nucleotide sequence having at least about 90% identity over an entire length of the nucleic acid sequence to a nucleotide sequence selected from the group consisting of SEQ ID NO:1 and SEQ ID NO:3;
b) a fragment of a nucleotide sequence having at least about 90% identity over at least 65% of the nucleic acid sequence to a nucleotide sequence selected from the group consisting of SEQ ID NO:1 and SEQ ID NO:3;
c) a nucleotide sequence selected from the group consisting of SEQ ID NO:1 and SEQ ID NO:3; and
d) a fragment of a nucleotide sequence comprising at least 65% of a nucleotide sequence selected from the group consisting of SEQ ID NO:1 and SEQ ID NO:3, and
wherein the second synthetic DNA encoded bispecific immune cell engager comprising an antigen binding domain targeting HER2 comprises a nucleotide sequence selected from the group consisting of:
e) a nucleotide sequence having at least about 90% identity over an entire length of the nucleic acid sequence to a nucleotide sequence selected from the group consisting of SEQ ID NO:5, and SEQ ID NO:7;
f) a fragment of a nucleotide sequence having at least about 90% identity over at least 65% of the nucleic acid sequence to a nucleotide sequence selected from the group consisting of SEQ ID NO:5, and SEQ ID NO:7;
g) a nucleotide sequence selected from the group consisting of SEQ ID NO:5, and SEQ ID NO:7; and
h) a fragment of a nucleotide sequence comprising at least 65% of a nucleotide sequence selected from the group consisting of SEQ ID NO:5, and SEQ ID NO:7.

18. A method of preventing or treating a disease or disorder in a subject, the method comprising administering to the subject a nucleic acid molecule of claim 1 or a composition comprising the same.

19. The method of claim 18, wherein the disease is selected from the group consisting of a benign tumor, cancer and a cancer-associated disease.

20. The method of claim 19, wherein the disease is glioblastoma.
